(19) United States
(12) Patent Application Publication
(10) Pub. No.: US 2002/0068827 A1
(43) Pub. Date: Jun. 6, 2002

(54) Quinoline-4-carboxamide derivatives, their preparation and their use as neurokinin 3 ( NK-3 ) - and neurokinin 2 ( NK-3 ) receptor antagonists

(75) Inventors: Giuseppe Arnaldo Maria Giardina, Milano (IT); Mario Grugni, Verbania (IT); Luca Francesco Raveglia, Milano (IT); Carlo Farina, Milano (IT)

Correspondence Address: SMITHKLINE BEECHAM CORPORATION, Corporate Intellectual Property - UW2220, P.O. Box 1539, King of Prussia, PA, 19406-0939, US

(73) Assignee: SmithKline Beecham S.p.A.

(21) Appl. No.: 09/994,402

(22) Filed: Nov. 26, 2001

Related U.S. Application Data
(63) Continuation of application No. 09/515,336, filed on Jun. 5, 2000, now abandoned.
Application No. 09/515,336 is a continuation of application No. 09/077,262, filed on Aug. 6, 1998, now abandoned.

(30) Foreign Application Priority Data
Nov. 24, 1995 (IT) MI95A002462
Aug. 2, 1996 (IT) MI96A001688
Nov. 22, 1996 (EP) PCT/EP96/05207

(51) Int. Cl. C07D 215/38; C07D 215/20; C07D 215/16

(52) U.S. Cl. 546/156; 546/159; 546/169

(57) ABSTRACT

A compound of formula (I):

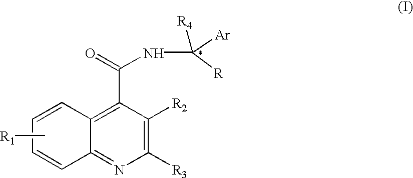

or a salt thereof, or a solvate thereof, wherein, Ar is an optionally substituted aryl or a C₅₋₇ cycloalkdienyl group, or an optionally substituted single or fused ring aromatic heterocyclic group;

R is C₁₋₆ alkyl, C₃₋₇ cycloalkyl, C₃₋₇ cycloalkylalkyl, optionally substituted phenyl or phenyl C₁₋₆ alkyl, an optionally substituted five-membered heteroaromatic ring comprising up to four heteroatoms selected from O and N, hydroxy C₁₋₆ alkyl, amino C₁₋₆ alkyl, C₁₋₆ alkylaminoalkyl, di C₁₋₆ alkylaminoalkyl, C₁₋₆ acylaminoalkyl, C₁₋₆ alkoxyalkyl, C₁₋₆ alkylcarbonyl, carboxy, C₁₋₆ alkoxycarbonyl, C₁₋₆ alkoxycarbonyl C₁₋₆ alkyl, aminocarbonyl, C₁₋₆ alkylaminocarbonyl, di C₁₋₆ alkylaminocarbonyl, halogeno C₁₋₆ alkyl; or R is a group —(CH₂)ₚ— wherein p is 2 or 3 which group forms a ring with a carbon atom of Ar;

R₁ represents hydrogen or up to four optional subtitutents selected from the list consisting of: C₁₋₆ alkyl, C₁₋₆ alkenyl, aryl, C₁₋₆ alkoxy, hydroxy, halogen, nitro, cyano, carboxy, carboxamido, sulphonamido, C₁₋₆ alkoxycarbonyl, trifluoromethyl, acyloxy, phthalimido, amino or mono- and di-C₁₋₆ alkylamino;

R₂ represents hydrogen, C₁₋₆-alkyl, hydroxy, halogen, cyano, amino, mono- or di-C₁₋₆-alkylamino, alkylsulphonylamino, mono- or di-C₁₋₆-alkanoylamino wherein any alkyl group is optionally substituted with an amino group or with a mono- or di-alkylamino group, or R₂ is a moiety —X—(CH₂)ₙ—Y wherein X is a bond or —O— and n is an integer in the range of from 1 to 5 providing that when X is —O— n is only an integer from 2 to 5 and Y represents a group NY₁Y₂ wherein Y₁ and Y₂ are independently selected from hydrogen, C₁₋₆-alkyl, C₁₋₆-alkenyl, aryl or aryl-C₁₋₆-alkyl or Y is hydroxy, halogen or an optionally substituted N-linked single or fused ring, heterocyclic group,

R₃ is branched or linear C₁₋₆ alkyl, C₃₋₇ cycloalkyl, C₄₋₇ cycloalkylalkyl, optionally substituted aryl, or an optionally substituted single or fused ring aromatic heterocyclic group; and

R₄ represents hydrogen or C₁₋₆ alkyl; a process for the preparation of such a compound, a pharmaceutical compositon containing such a compound and the use of such a compound or composition in medicine.

The present invention relates to novel compounds, in particular to novel quinoline derivatives, to processes for the preparation of such compounds, to pharmaceutical compositions containing such compounds and to the use of such compounds in medicine.

The mammalian peptide Neurokinin B (NKB) belongs to the Tachykinin (TK) peptide family which also include Substance P (SP) and Neurokinin A (NKA). Pharmacological and molecular biological evidence has shown the existence of three subtypes of TK receptor (NK₁, NK₂ and NK₃) and NKB binds preferentially to the NK₃ receptor although it also recognises the other two receptors with lower affinity (Maggi et al, 1993, J. Auton. Pharmacol., 13, 23-93).

Selective peptidic NK₃ receptor antagonists are known (Drapeau, 1990 Regul. Pept. 31, 125-135), and findings with peptidic NK₃ receptor agonists suggest that NKB, by activating the NK₃ receptor, has a key role in the modulation of neural input in airways, skin, spinal cord and nigro-striatal pathways (Myers and Undem. 1993, J.Physiol., 470, 665-679; Counture et al., 1993, Regul. Peptides. 46, 426-429; Mccarson and Krause, 1994, J. Neurosci., 14 (2), 712-720; Arenas et al. 1991, J.Neurosci., 11, 2332-8). However, the peptide-like nature of the known antagonists makes them likely to be too labile from a metabolic point of view to serve as practical therapeutic agents.

We have now discovered a novel class of non-peptide NK-3 antagonists which are far more stable from a metabolic point of view than the known peptidic NK-3 receptor antagonists and are of potential therapeutic utility. These compounds also have NK-2 antagonist activity and are therefore considered to be of potential use in the prevention and treatment of a wide variety of clinical conditions which are characterized by overstimulation of the tachvkinin receptors, in particular NK-3 and NK-2.

These conditions include respiratory diseases, such as chronic obstructive pulmonary disease (COPD), asthma, airway hyperreactivity, cough; inflammatory diseases such as inflammatory bowel disease, psoriasis, fibrositis, osteoarthritis, rheumatoid arthritis and inflammatory pain; neurogenic inflammation or peripheral neuropathy, allergies such as eczema and rhinitis; ophthalmic diseases such as ocular inflammation. conjunctivitis, vernal conjuctivitis and the like; cutaneous diseases, skin disorders and itch, such as cutaneous wheal and flare, contact dermatitis, atopic dermatitis, urticaria and other eczematoid dermatitis; adverse immunological reactions such as rejection of transplanted tissues and disorders related to immune enhancement or suppression such as systhemic lupus erythematosis; gastrointestinal (GI) disorders and diseases of the GI tract such as disorders associated with the neuronal control of viscera such as ulcerative colitis, Crohn's disease and urinary incontinence; renal disorders and disorders of the bladder function, (hereinafter referred to as the ‘Primary Conditions’).

Certain of these compounds also show CNS activity and hence are considered to be of particular use in the treatment of disorders of the central nervous system such as anxiety, depression, psychosis and schizophrenia; neurodegenerative disorders such as AIDS related dementia, senile dementia of the Alzheimer type, Alzheimer's disease, Down's syndrome, Huntington's disease, Parkinson's disease, movement disorders and convulsive disorders (for example epilepsy); demyelinating diseases such as multiple sclerosis and amyotrophic lateral sclerosis and other neuropathological disorders such as diabetic neuropathy, AIDS related neuropathy, chemotherapy-induced neuropathy and neuralgia; addiction disorders such as alcoholism; stress related somatic disorders; reflex sympathetic dystrophy such as shoulder/hand syndrome; dysthymic disorders; eating disorders (such as food intake disease); fibrosing and collagen diseases such as scleroderma and eosinophilic fascioliasis; disorders of the blood flow caused by vasodilation and vasospastic diseases such as angina, migraine and Reynaud's disease and pain or nociception, for example, that is attributable to or associated with any of the foregoing conditions especially the transmission of pain in migraine, (hereinafter referred to as the ‘Secondary Conditions’).

The compounds of formula (I) are also considered to be useful as diagnostic tools for assessing the degree to which neurokinin-3 receptor activity (normal, overactivity or underactivity) is implicated in a patient's symptoms.

According to the present invention there is provided a compound, or a solvate or a salt thereof, of formula (I):

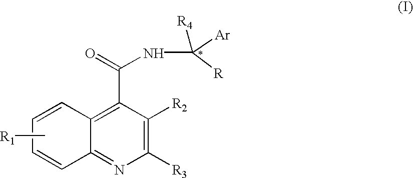

wherein, Ar is an optionally substituted aryl or a C₅₋₇ cycloalkdienyl group, or an optionally substituted single or fused ring aromatic heterocyclic group;

R is C₁₋₆ alkyl, C₃₋₇ cycloalkyl, C₃₋₇ cycloalkylalkyl, optionally substituted phenyl or phenyl C₁₋₆ alkyl, an optionally substituted five-membered heteroaromatic ring comprising up to four heteroatoms selected from O and N, hydroxy C₁₋₆ alkyl, amino C₁₋₆ alkyl, C₁₋₆ alkylaminoalkyl. di C₁₋₆ alkylaminoalkyl, C₁₋₆ acylaminoalkyl, C₁₋₆ alkoxyalkyl, C₁₋₆ alkylcarbonyl, carboxy, C₁₋₆ alkoxycarbonyl, C₁₋₆ alkoxycarbonyl C₁₋₆ alkyl, aminocarbonyl, C₁₋₆ alkylaminocarbonyl, di C₁₋₆ alkylaminocarbonyl, halogeno C₁₋₆ alkyl; or R is a group —(CH₂)ₚ— wherein p is 2 or 3 which group forms a ring with a carbon atom of Ar;

R₁ represents hydrogen or up to four optional subtitutents selected from the list consisting of: C₁₋₆ alkyl, C₁₋₆ alkenyl, aryl, C₁₋₆ alkoxy, hydroxy, halogen, nitro, cyano, carboxy, carboxamido, sulphonamido. C₁₋₆ alkoxycarbonyl, trifluoromethyl, acyloxy, phthalimido, amino or mono- and di-C₁₋₆ alkylamino;

R₂ represents hydrogen, C₁₋₆-alkyl, hydroxy, halogen, cyano, amino, mono- or di-C₁₋₆-alkylamino, alkylsulphonylamino, mono- or di-C₁₋₆-alkanoylamino wherein any alkyl group is optionally substituted with an amino group or with a mono- or di-alkylamino group, or R₂ is a moiety —X—(CH₂)ₙ—Y wherein X is a bond or —O— and n is an integer in the range of from I to 5 providing that when X is —O— n is only an integer from 2 to 5 and Y represents a group NY₁Y₂ wherein Y₁ and Y₂ are independently selected from hydrogen, C₁₋₆-alkyl, C₁₋₆-alkenyl, aryl or aryl-C₁₋₆-alkyl or Y is hydroxy, halogen or an optionally substituted N-linked single or fused ring, heterocyclic group, R₃ is branched or linear C₁₋₆ alkyl, C₃₋₇ cycloalkyl, C₄₋₇ cycloalkylalkyl, optionally substituted aryl, or an optionally substituted single or fused ring aromatic heterocyclic group; and

R₄ represents hydrogen or C₁₋₆ alkyl.

Suitably, Ar represents optonally substituted phenyl, preferably unsubstituted phenyl.

When R represents C₁₋₆ alkylcarbonyl, an example is acetyl.

When R represents C₁₋₆ alkoxycarbonyl, an example is methoxycarbonyl.

Suitably, R represents CI alkyl, for example ethyl.

Preferably, R is ethyl.

Suitably, R₁ represents hydrogen or C₁₋₆ alkyl for example methyl.

Preferably, R₁ is hydrogen.

When R₂ represents halogen it is suitably fluorine.

When R₂ represents mono- or di-C₁₋₆-alkanoylamino, the alkanoyl group is favourably an N-hexanoyl group suitably substituted with an amino group on the terminal carbon atom.

When Y is an optionally substituted N-linked single or fused heterocyclic group, any single or fused ring is suitably saturated or unsaturated and consisting of 5- or 6-ring atoms, said ring atoms optionally comprising 1 or 2 additional heteroatoms selected from O or N.

When Y is an N-linked single or fused heterocyclic group, one or two ring atoms are optionally substituted with one or two oxo groups or one or two hydroxy, C₁₋₆ alkoxycarbonyl, C₁₋₆ alkyl, aryl or a single or fused ring aromatic heterocyclic group, or the substituents on adjacent ring atoms form a carbocyclic ring; said aryl or aromatic heterocyclic groups being optionally substituted with one or two C₁₋₆ alkyl, alkoxy, hydroxy, halogen or halogenalkyl groups.

Preferably, Y represents an N-linked single or fused heterocyclic group, any single or fused ring being saturated or unsaturated and consisting of 5- or 6-ring atoms, said ring atoms optionally comprising 1 or 2 additional heteroatoms selected from O or N and wherein one or two ring atoms are optionally substituted with one or two oxo groups or one or two hydroxy, C₁₋₆ alkoxycarbonyl, C₁₋₆ alkyl aryl or a single or fused ring aromatic heterocyclic group, or the substituents on adjacent ring atoms form a carbocyclic ring; said aryl or aromatic heterocyclic groups being optionally substituted with one or two C₁₋₆ alkyl, alkoxy, hydroxy, halogen or halogenalkyl groups.

When Y represents the above mentioned heterocyclic group having an OH or an oxo substituent on one or two of the ring atoms, said atoms are preferably positioned adjacent to the linked N atom.

A suitable N-linked single ring 6-membered saturated heterocyclic group comprising an additional heteroatom is a morpholino group or a piperizinyl group, for example an optionally substituted 4-phenylpiperazinyl group.

Suitable N-linked fused ring heterocyclic groups comprise a 5-or 6-membered saturated or unsaturated heterocyclic ring fused to a benzene ring.

A suitable N-linked fused ring heterocyclic group comprising a 6-membered saturated heterocyclic ring fused to a benzene ring is a 2-(1,2,3,4-tetrahydro)isoquinolinyl group.

A suitable N-linked fused ring heterocyclic group comprising a 5-membered saturated heterocyclic ring fused to a benzene ring is a 2-isoindolinyl group.

A suitable N-linked fused ring heterocyclic group comprising a 6-membered unsaturated heterocyclic ring fused to a benzene ring and having an oxo substituent on one saturated ring atom is a 1,4-dihydro-3(2H)-isoquinolinon-2-yl group or a 3,4-dihydro-1(2H)-isoquinolinon-2-yl group.

A suitable N-linked fused ring heterocyclic group comprising a 6-membered unsaturated heterocyclic ring fused to a benzene ring and having an oxo substituent on two saturated ring carbon atoms is an homophthalimido group.

When R₂ represents a moiety —(CH₂)ₙ—Y, examples of Y include an amino group or a mono- or di-C₁₋₆-alkylamino group. A further example of Y in the moiety —(CH₂)ₙ—Y is a morpholino group or a 4-phenylpiperazine group or an N-methyl-N-benzylamino group.

A preferred value for the moiety —X—(CH₂)ₙ—Y is a moiety of formula (a):

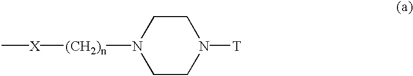

wherein T represents C₁₋₆ alkyl, C₁₋₆ alkoxycarbonyl, aryl or an aromatic heterocyclic group and either X is O and n is 2 or 3 or X is a bond and n is 1, 2 or 3.

Suitably X is O. Suitably X is a bond.

When T represents a C₁₋₆ alkyl group, it is preferably a methyl group.

When T represents an aryl group it is suitably an optionally substituted phenyl group, preferably a phenyl group substituted with one or more, for example up to 3, alkoxy groups, especially methoxy groups, especially when substituted at position 2 relative to the point of attachment on the piperazinyl group.

When T represents an aromatic heterocyclic group, a suitable group is a 6 membered aromatic heterocyclic group having 2 nitrogen atoms, suitably a pyrimidine group and preferably a 2-pyrimidine group.

A further preferred value for the moiety —X—(CH₂)ₙ—Y is a moiety of formula (b):

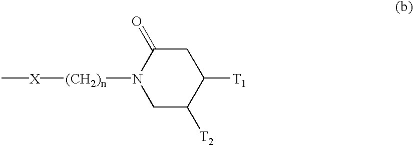

wherein X is O or a bond, n is 1, 2 or 3, T₁ and T₂ each independently represents hydroxy, C₁₋₆ alkoxycarbonyl, C₁₋₆ alkyl, aryl or a single or fused ring aromatic heterocyclic group, or T₁ and T₂ together with the carbon atoms to which they are attached form a carbocyclic ring; said aryl or aromatic heterocyclic groups being optionally substituted with one or two C₁₋₆ alkyl, alkoxy, hydroxy, halogen, halogenalkyl groups; or one of T₁ or T₂ is an oxo group and the other is selected from the above mentioned groups as appropriate.

Preferably, T₁ and T₂ together with the carbon atoms to which they are attached form a carbocyclic ring, in particular a cyclohexyl ring.

When R₂ represents a moiety —(CH₂)ₙ—Y, n is suitably an integer 1 or 2, for example 1.

Examples of the moiety —(CH₂)ₙ—Y include aminomethyl and methylaminomethyl, a further example is morpholinomethyl.

When R₂ represents a moiety —O—(CH₂)ₙ—Y, examples of Y include OH, —2-isoindolinyl, homophthalimido, -2-(1,2,3,4-tetrahydro)isoquinolinyl, 1,4-dihydro-3(2H)-isoquinolinon-2-yl and, especially, 3,4-dihydro-1(2H)-isoquinolinon-2-yl. Further examples of Y in the moiety O—(CH₂)ₙ—Y are: phthalimido; 3-hydroxy-3,4-dihydro-1(2H)-isoquinolinon-2-yl; 1-(2H)-isoquinolinon-2-yl (a favoured group); succinimido; maleimido; 2.2-dimethyl-4-oxo-3-imidazolidinyl; 4-(2-methoxyphenyl) piperazin-1-yl (a favoured group); 4-(3-chlorophenyl)piperazin-1-yl (a favoured group); 4-phenylpiperazin-1-yl (afavoured group), 4-(2-pyrimidinyl)piperazin-1-yl (a favoured group); 2-phenyl-4-oxo-3-imidazolidinyl and 2,2-dimethyl-5-phenyloxo-3-imidazolidinyl.

When R₂ represents a moiety —O—(CH₂)ₙ—Y, n is suitably an integer 2 or 3.

Preferably, R₂ represents a moiety —X—(CH₂)ₙ—Y.

In one aspect X is a bond.

Suitably, X represents O. When R₄ is C₁₋₆ alkyl, an example is methyl.

Preferred compounds of formula (I) are those wherein:

Ar is phenyl, R is ethyl, R₁ is hydrogen, R₂ is a moiety —X—(CH₂)ₙ—Y wherein X is, preferably, O or a bond, n is 1, 2 or 3 and Y is a moiety formula (a) or (b) as defined above; in particular should be mentioned the compounds of examples 18, 30, 33 and 40.

The compounds of formula (I) may have at least one asymmetric centre—for example the carbon atom labelled with an asterisk (*) in the compound of formula (I)—and therefore may exist in more than one stereoisomeric form. The invention extends to all such stereoisomeric forms and to mixtures thereof, including racemates. In particular, the invention includes compounds wherein the asterisked carbon atom in formula (I) has the stereochemistry shown in formula (Ia):

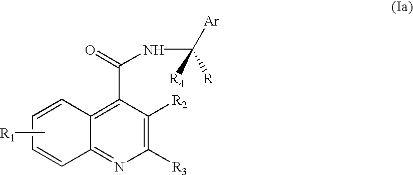

wherein Ar, R, R₁, R₂, R₃, and R₄ are as defined in relation to formula (I).

The compounds of formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels.

A substantially pure form will generally contain at least 50% (excluding normal pharmaceutical additives), preferably 75%, more preferably 90% and still more preferably 95% of the compound of formula (I) or its salt or solvate.

One preferred pharmaceutically acceptable form is the crystalline form, including such form in pharmaceutical composition. In the case of salts and solvates the additional ionic and solvent moieties must also be non-toxic.

Suitable salts are pharmaceutically acceptable salts.

Suitable pharmaceutically acceptable salts include the acid addition salts with the conventional pharmaceutical acids, for example maleic, hydrochloric, hydrobromic, phosphoric, acetic, fumaric, salicylic, citric, lactic, mandelic, tartaric, succinic, benzoic, ascorbic and methanesulphonic.

Suitable pharmaceutically acceptable salts include salts of acidic moieties of the compounds of formula (I) when they are present, for example salts of carboxy groups or phenolic hydroxy groups.

Suitable salts of acidic moieties include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-p-phenethylamine, dehydroabietylamine, N,N′-bisdehydroabietylamine, glucamine, N-methylglucamine or bases of the pyridine type such as pyridine. collidine, quinine or quinoline.

Suitable solvates are pharmaceutically acceptable solvates.

Suitable pharmaceutically acceptable solvates include hydrates.

The term ‘alkyl’ (unless specified to the contrary) when used alone or when forming part of other groups (such as the ‘alkoxy’ group) includes straight- or branched-chain alkyl groups containing 1 to 12 carbon atoms, suitably 1 to 6 carbon atoms, examples include methyl, ethyl, n-propyl, isopropyl, n-butyl, isobutyl or tert-butyl group.

The term ‘carbocylic’ refers to cycloalkyl and aryl rings.

The term ‘cycloalkyl’ includes groups having 3 to 12, suitably 4 to 6 ring carbon atoms.

The term ‘aryl’ includes phenyl and naphthyl, preferably phenyl which unless specified to the contrary optionally comprise up to five, preferably up to three substituents selected from halogen, alkyl, phenyl, alkoxy, haloalkyl, hydroxyalkyl, hydroxy, amino, nitro, cyano, carboxy, alkoxycarbonyl, alkoxycarbonylalkyl, alkylcarbonyloxy, or alkylcarbonyl groups.

The term ‘aromatic heterocyclic group’ includes groups comprising aromatic heterocyclic rings containing from 5 to 12 ring atoms, suitably 5 or 6, and comprising up to four hetero-atoms in the or each ring selected from S, O or N.

Unless specified to the contrary, suitable substituents for any heterocyclic group includes up to 4 substituents selected from the group consisting of: alkyl, alkoxy, aryl and halogen or any two substituents on adjacent carbon atoms, together with the carbon atoms to which they are attached, may form an aryl group, preferably a benzene ring, and wherein the carbon atoms of the aryl group represented by the said two substituents may themselves be substituted or unsubstituted.

When used herein the term “halogen” refers to fluorine, chlorine, bromine and iodine, preferably fluorine or chlorine.

When used herein the term “acyl” includes residues of acids, in particular a residue of a carboxylic acid such as an alkyl- or aryl-carbonyl group.

The invention also provides a process for the preparation of a compound of formula (I), or a salt thereof and/or a solvate thereof, which process comprises reacting a compound of formula (III):

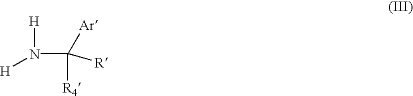

wherein R′, R₄′ and Ar′ are R, R₄ and Ar as defined for formula (I) or a group or atom convertible to R, R₄ and Ar respectively, with a compound of formula (II) or an active derivative thereof:

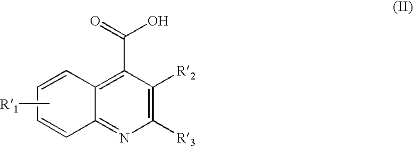

wherein R′₁, R′₂ and R′₃ are R₁, R₂ and R₃ respectively as defined in relation to formula (I) or a group convertible to R₁, R₂ and R₃ to form a compound of formula (Ib):

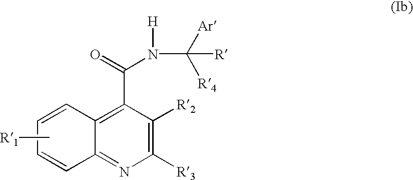

wherein Ar′, R′, R′₁, R′₂, R′₃and R′₄ are as defined above, and optionally thereafter carrying out one or more of the following optional steps:

(i) converting any one of Ar′, R′, R′₁, R′₂, R′₃ and R′₄ to Ar, R, RI, R₂I , R₃ or R₄ respectively as required, to obtain a compound of formula (I);

(ii) converting a compound of formula (I) into another compound of formula (I); and

(iii) preparing a salt of the compound of formula (I) and/or a solvate thereof.

Suitable groups convertible into other groups include protected forms of said groups.

Suitably Ar′, R′, R′₁ or R′₃ each represents Ar, R, R₁, or R₃ respectively or a protected form thereof.

Suitably R′₂ represents a group other than a protected form which is convertible into R₂ by conventional procedures.

Suitably, R′₄ represents hydrogen, so that compounds of formula (I) wherein the required R₄ is alkyl are conveniently prepared from the corresponding compound wherein R₄ is hydrogen.

It is favoured if the compound of formula (II) is present as an active derivative.

A suitable active derivative of a compound of formula (II) is a transient activated form of the compound of formula (II) or a derivative wherein the carboxy group of the compound of formula (II) has has been replaced by a different group or atom, for example by a carboxy halide, preferably a chloride, or an azide or a carboxylic acid anhydride.

Other suitable active derivatives include: a mixed anhydride formed between the carboxyl moiety of the compound of formula (II) and an alkyl chloroformate; an activated ester, such as a cyanomethyl ester, thiophenyl ester, p-nitrophenyl ester, p-nitrothiophenyl ester, 2,4,6-trichlorophenyl ester, pentachlorophenyl ester, pentafluorophenyl ester, N-hydroxy-phtalimido ester, N-hydroxypiperidine ester, N-hydroxysuccinimide ester, N-hydroxy benzotriazole ester; alternatively, the carboxy group of the compound of formula (II) may be activated using a carbodiimide or N,N′-carbonyldiinidazole.

The reaction between the compound of formula (II) or the active derivative thereof and the compound of formula (III) is carried out under the appropriate conventional conditions for the particular compounds chosen. Generally, when the compound of formula (II) is present as an active derivative the reaction is carried out using the same solvent and conditions as used to prepare the active derivative, preferably the active derivative is prepared in situ prior to forming the compound of formula (Ib) and thereafter the compound of formula (I) or a salt thereof and/or a solvate thereof is prepared.

For example, the reaction between an active derivative of the compound of formula (II) and the compound of formula (III) may be carried out:

(a) by first preparing an acid chloride and then coupling said chloride with the compound of formula (III) in the presence of an inorganic or organic base in a suitable aprotic solvent such as dimethylformamide (DMF) at a temperature in a range from −70 to 50° C. (preferably in a range from −10 to 20° C.); or

(b) by treating the compound of formula (II) with a compound of formula (III) in the presence of a suitable condensing agent, such as for example N,N′-carbonyl diimidazole (CDI) or a carbodiimide such as dicyclohexylcarbodiimide (DCC) or N-dimethylaminopropyl-N′-ethylcarbodiimide, preferably in the presence of N-hydroxybenzotriazole (HOBT) to maximise yields and avoid racemization processes (see Synthesis, 453, 1972), in an aprotic solvent, such as a mixture of acetonitrile (MeCN) and tetrahydrofuran (THF), for example a mixture in a volume ratio of from 1:9 to 7:3 (MeCN:THF), at a temperature in the range of from −70 to 50° C. (preferably in a range of from −10 to 25° C.).

A preferred reaction is set out in Scheme 1 shown below:

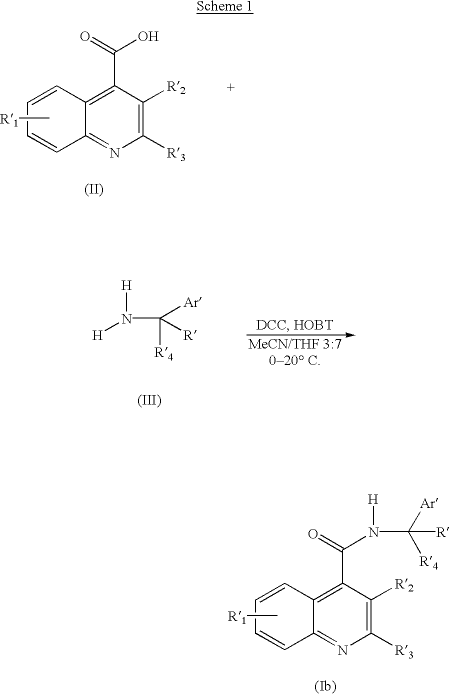

wherein Ar′, R′, R′₁, R′₂, R′₃ and R′₄ are as defined above. It will be appreciated that a compound of formula (Ib) may be converted to a compound of formula (I), or one compound of formula (I) may be converted to another compound of formula (I) by interconversion of suitable substituents. Thus, certain compounds of formula (I) and (Ib) are useful intermediates in forming other compounds of the present invention.

Accordingly, in a further aspect the invention provides a process for preparing a compound of formula (I), or a salt thereof and/or a solvate thereof, which process comprises converting a compound of the above defined formula (Ib) wherein at least one of Ar′, R′, R′₁ R′₂, R′₃ or R′₄ is not Ar, R, R₁, R₂, R₃ or R₄ respectively, thereby to provide a compound of formula (I); and thereafter, as required, carrying out one or more of the following optional steps:

(i) converting a compound of formula (I) into another compound of formula (I); and

(ii) preparing a salt of the compound of formula (I) and/or a solvate thereof.

Suitably, in the compound of formula (Ib) the variables Ar′, R′, R′₁ and R′₃ are Ar, R, R₁ or R₃ respectively or they are protected forms thereof, R′₂ is a group or atom which may be converted into a variable R₂ by one or more steps and R′₄ is hydrogen which thereafter is converted as required into a C₁-6 alkyl group.

Favourably, R′₂ represents OH, CH₃ or an amino group.

R′₂ can also represent a moiety —X—(CH₂)ₙ—Y′ wherein X and n are as defined in relation to the compounds of formula (I) and Y′ is a group Y which is convertible into another group Y, for example Y′ represents NH₂.

The conversion of any group Ar′, R′, R′, or R′₃ into Ar, R, R₁ or R₃, which as stated above are usually protected forms of Ar, R, R₁ or R₃ may be carried out using appropriate conventional conditions such as the appropriate deprotection procedure.

The conversion of any group R′₂ into R₂ (including the conversion of any group Y′ into another group Y in the above mentioned moiety —X—(CH₂)ₙ—Y′) may be carried out using appropriate conventional reagents and conditions:

For example, when R′₂ is OH, the compounds of formula (Ib) can be converted to compounds of formula (I) as described in Schemes 2a and 2b.

[chemical structure]

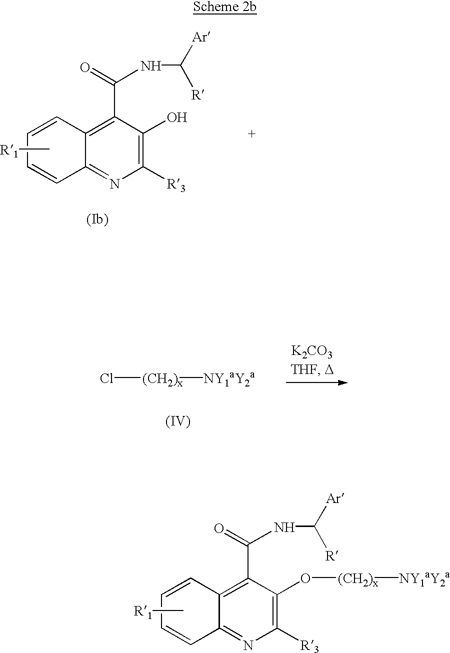

wherein Ar′, R′, R′₁, R′₂, and R′₃ are as defined above, LI is a leaving group or atom, such as a halogen atom for example bromine, L₂, and L₃ each independently represent a leaving group or atom, preferably the same leaving group or atom, such as a halogen atom for example bromine, q is an integer 1 or 2, r is zero or an integer 1, x is an integer in the range of from 2 to 5, y is an integer in the range of from 1 to 4, Y₁a and Y₂a together with the nitrogen to which they are attached represent an N-linked single or fused ring heterocyclic group, any single or fused ring being saturated or unsaturated and consisting of 5- or 6-ring atoms, said ring atoms optionally comprising 1 or 2 additional heteroatoms selected from O or N and wherein one or two ring atoms are optionally substituted with one or two oxo groups or one or two hydroxy, C₁₋₆ alkoxycarbonyl, C₁₋₆ alkyl, aryl or a single or fused ring aromatic heterocyclic groups, said aryl or aromatic heterocyclis groups being optionally substituted with one or two C₁₋₆ alkyl, alkoxy, hydroxy, halogen, halogenalkyl groups.

In Scheme 2a as illustrated, an example of HN Y₁^{a}Y₂^{a} is 1,2,3,4-tetrahydroisoquinoline.

The reactions in Schemes 2a and 2b illustrate that when R₂′ is OH the compound of formula (Ib) can be converted into a compound wherein R₂ is —O—(CH₂)ₙ—Y′ wherein n is as defined in relation to the compounds of formula (I) and Y′ is Y as defined in relation to formula (I) or is a group convertible thereto, by reaction with a compound of formula (IV):

L1—(CH₂)ₙ—Y′ (IV)

wherein n and Y′ are as defined and illustrated above and L₁ is a leaving group or atom, such as a halogen atom, for example bromine and chorine.

The particular reaction conditions used depends upon such factors as the specific nature of the required conversion and the nature of the compound of formula (IV) but generally the appropriate conventional conditions are employed. For example:

As is shown in Scheme 2a, when R′₂ is OH, it can be converted to 2-aminoalkoxy by reaction with 2-bromoalkylphthalimide and potassium carbonate (K₂CO₃) in boiling THF to obtain the phthalimido derivative which is, in turn, hydrolized with hydrazine hydrate in alcoholic medium.

The primary amine (i.e. when R′₂ is O(CH₂)ₙ NH₂ wherein n is as defined above) can be converted to a cyclic tertiary amine by reacting with an o-dibromoalkyl benzene in DMF at 80° C., using TEA to trap the forming hydrogen bromide. The primary aminoalkoxy quinoline can also be tranformed in an homophthalimidoalkoxy quinoline, by refluxing with homophthalic anhydride in toluene, azeotroping the forming water with a Dean-Starck apparatus or using 4 Å molecular sieves. The carbonyl at position 3 of the homophthalimido group can be reduced to hydroxy with sodium borohydride NaBH₄) in methanol at room temperature; subsequently, the hydroxy group can be eliminated by reaction with mesyl chloride (MSCl) and TEA and the forming double bond can be reduced with hydrogen using a palladium on carbon catalyst (5% Pd on C) in a mixture of acetic acid and trifluoroacetic acid (AcOH/TFA).

The hydroxy group at position 3 of the quinoline ring can also be alkylated with a bromoalkyl ester, for example ethyl bromoacetate, and K₂CO₃ in THF at room temperature, the resulting ester moiety can be reduced to alcohol with a selective metal borohydride, such as NaBH₄ in boiling t-BuOHMeOH (Bull. Chem. Soc. Japan, 1984, 57, 1948 or Synth. Commun., 1982, 12, 463). The hydroxy moiety may then be oxidized to the corresponding aldehyde in standard Swern conditions, with oxalyl chloride/DMSO at −60° C. in CH₂Cl₂ (Tetrahedron, 1978, 34, 1651). Reductive amination of the so formed aldehyde with a cyclic secondary amine, such as 1,2,3,4-tetrahydroisoquinoline and NaCNBH₃ in methanol at room temperature (J. Am. Chem. Soc., 1971, 93, 2897) affords the corresponding 1,2,3,4-tetrahydroisoquinolinylalkoxy derivative.

In Scheme 2 bit is illustrated that the compound of formula (Ib) wherein R₂′ is OH can be reacted with a compound of formula (IV) wherein Y is an N-linked single or fused ring heterocyclic group as defined in relation to Y of formula (I), to provide the respective compound of formula (I) wherein Y is the said N-linked single or fused ring heterocyclic group. In Scheme 2b the heterocyclic group HNY₁^{a}Y₂^{a} is, for example, an N linked piperazine. The reaction is carried out using conventional alkylation conditions in an aprotic solvent such as tetrahydrofuran, preferably in the presence of a base, for example potassium carbonate, usually at an elevated temperature, conveniently at the reflux temperature of the solvent.

When R′₂ is CH₃, compounds (Ib) can be converted to other compounds of formula (I) as described in Scheme 3.

[chemical structure]

wherein Ar′, R′, R′₁, R′₂ and R′₃ are as defined above and wherein Y₁^{a} and Y₂^{a} are as defined in relation to Scheme 2a or 2b.

In particular, when R′₂ is CH₃, it can be transformed to a (monoalkyl) or (dialkyl) aminomethyl quinoline derivative by reacting the intermediate bromomethyl derivative (prepared using N-bromosuccinimide in dichloroethane in the presence of a catalytic amount of benzoylperoxide) with the appropriate amines, to yield, for example the 3-morpholinomethyl derivative.

When R′₂ is NH₂, compounds (lb) can be converted to other compounds of formula (I) using the appropriate conventional procedures.

In particular, when R′₂ is NH₂, it can be converted to a (monoalkyl) or (dialkyl)amino acylamino group by reaction with an ω-chloroacylchloride and subsequent displacement of the chlorine atom or with potassium phthalimide in refluxing DMF, followed by hydrolisis with hydrazine hydrate in alcoholic medium, or with the appropriate mono- or di-alkylamine in methanol as solvent at a temperature from 20° to 100° C.

In a further particular aspect, there is provided a process for the preparation of compounds of formula (I) wherein Ar is phenyl, R is C₁₋₆ alkyl, R₄ is hydrogen or C₁₋₆ alkyl and R₂ represents a moiety —(CH₂)ₙ—NHY₃ wherein Y₃ is a group —CR(Ar)(R₄) wherein Ar and R are as last above defined and n is as defined in relation to formula (I), which process comprises:

(a) halogenating a compound of formula (II) wherein R′₁ and R′₃ are as defined above and R′₂ is —(CH₂)ₙ-1-CH₃; and thereafter

(b) reacting the halogenated product with a compound of formula (V):

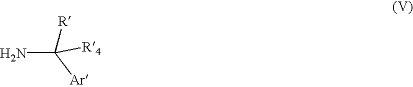

wherein Ar′, R′ and R′₄ are as last above defined or are protected forms thereof.

The compound of formula (II) is preferably in an activated form, as described above, and especially as a tert butyl ester.

The halogenation reaction is effected by use of conventional halogenating reagents, such as the use of N-bromosuccinamide for bromination usually in an inert solvent such as carbon tetrachloride, at any temperature providing a convenient rate of formation of the required product, suitably at an elevated temperature such as the reflux temperature of the solvent.

The reaction between the said halogenated product, and the compound of formula (V) is suitably carried out in a protic solvent, usually an alkanolic solvent such as ethanol, at a temperature in the range of from 0° C. to 50° C.

The conversion of R′₄ when representing hydrogen into a C₁₋₆ alkyl group is carried out using the appropriate conventional procedure, for example the procedure shown in Scheme 4:

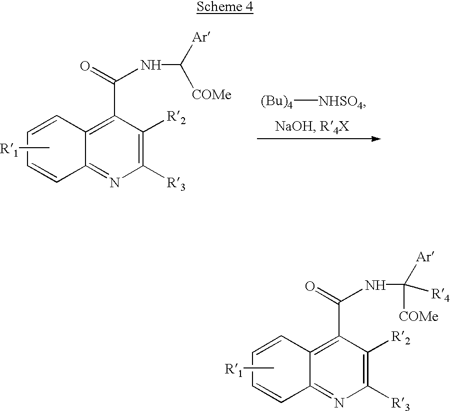

wherein Ar′, R′, R′₁, R′₂, R′₃ and R′₄ are as defined above.

Suitable conversions of one compound of formula (I) into another compound of formula (I) include conversions wherein one group R, R₁, R₂, R₃ or R₄ is converted into another group R, R₁, R₂, R₃ or R₄ respectively, said conversions conveniently proceeding via appropriate groups Ar′, R′, R′₁, R′₂, R₃ and R′₄ using conventional methodology, for example those methods described in the reaction Schemes herein.

Examples of conversions of one compound of formula (I) into another compound of formula (I) include those wherein R₂ is converted into other values of R₂. Thus when R₂ is a group —O—(CH₂)ₙ—NH₂ wherein n is as defined in relation to formula (I) suitable conversions into other values of R₂ are illustrated in Scheme 5:

[chemical structure]

wherein Ar′, R′, R₁′, R₂ and R₃′ are as defined in relation to the compounds of formulae (II) and (III).

The reaction of the compound of formula (I) wherein R₂ is a group —O—(CH₂)ₙ—NH₂ (the ‘primary amine’) with FMOC protected glycinyl chloride or an appropriately substituted derivative thereof to provide a compound having an N-linked 4-oxoimidazolidinyl group, or a substituted derivative thereof, is conveniently carried out in an inert solvent such as methylene dichloride at any temperature providing a convenient rate of formation of the required product, usually at reduced to ambient temperature, for example in the range of 0° C. to ambient temperature to initially provide an aminoacetylaminoethoxy intermediate or an appropriately substituted derivative thereof. Ring closure of this intermediate is effected by treatment with an appropriate aldehyde or ketone depending upon the nature of the required ring. Thus, when the required ring is a 2,2-dimethyl substituted ring then acetone is used, usually in an n-butanol solvent at reflux, or when a 2-phenyl substituted ring is required then benzaldehyde is used, in refluxing methanol.

Alternatively, when the primary amine intermediate is reacted with succinic anhydride in an aromatic hydrocarbon solvent such as toluene, usually at an elevated temperature, for example the reflux temperature of the solvent, the 3-carboxypropanoyl intermediate produced can be cyclised to provide a succinamido group by heating with tetrahydronaphthaline.

A compound wherein Y is a 1,4-dihydro-3(2H)-isoquinolinon-2-yl group or a derivative thereof is prepared from the primary amine intermediate by reaction with an appropriate isochromanone in an alkanolic solvent, such as ethanol suitably absolute ethanol, at an elevated temperature such as the reflux temperature of the solvent to provide a 2-(2-hydroxymethyl)phenylacetyl intermediate which is cyclised first by activation, for example by chlorinating the hydroxymethyl group with thionyl chloride, followed by treatment with a base such as sodium hydride in tetrahydrofuran to effect cyclisation; preferably the cyclisation carried out in the presence of a catalytic amount of 1,3-dimethyl-2-imidazolidinone.

As mentioned before, the compounds of formula (I) may exist in more than one stereoisomeric form and the process of the invention may produce racemates as well as enantiomerically pure forms. Accordingly, a pure enantiomer of a compound of formula (1) is obtained by reacting a compound of the above defined formula (II) with an appropriate enantiomerically pure primary amine of formula (IIIa) or (IIIc):

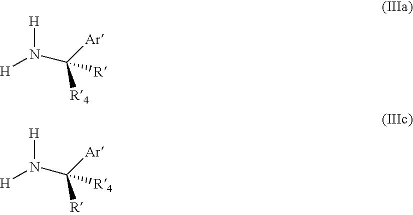

wherein R′, R′₄and Ar′ are as defined above, to obtain a compound of formula (I′a) or (I′c):

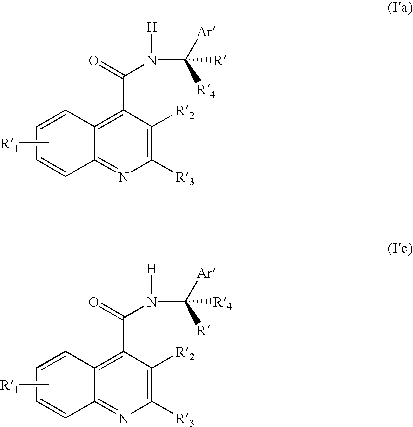

wherein Ar′, R′, R′₁, R′₂, R′₃ and R′₄ are as defined above.

Compounds of formula (I′a) or (I′c) may subsequently be converted to compounds of formula (Ia) or (Ic) by the methods of conversion mentioned before:

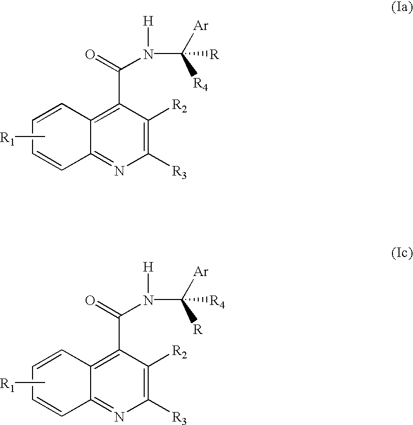

wherein Ar, R, R₁ R₂, R₃ and R₄ are as defined above.

Suitably, in the above mentioned compounds of formulae (Ia), (Ic), (I′a), (I′c), (III′a) and (III′c) R₄ represents hydrogen.

An alternative method for separating optical isomers, for example for those compounds of formula (I) wherein R₄ is different from hydrogen, is to use conventional, fractional separation methods in particular fractional crystallization methods. Thus, a pure enantiomer of a compound of formula (I) is obtained by fractional crystallisation of a diastereomeric salt formed by reaction of the racemic compound of formula (I) with an optically active strong acid resolving agent, such as camphosulphonic acid, in an appropriate alcoholic solvent, such as ethanol or methanol, or in a ketonic solvent, such as acetone. The salt formation process should be conducted at a temperature between 20° C. and 80° C., preferably at 50° C.

In the case in which other basic functionalities, such as primary, secondary or tertiary amine, are present in the molecule, a wider range of optically active acid resolving agents become available, including tartaric acid, O,O′-di-p-toluoyltartaric acid and mandelic acid.

The compounds of formula (II) wherein R, is CH₃, OH or NH₂ and protected forms of such compounds are either known compounds or they are prepared according to methods used to prepare known compounds, for example 3-methyl-2-phenyl-4-quinoline carboxylic acid (R₂ is CH₃, CAS=[43071-45-0]) is prepared in accordance with the methods described in Synthesis (1993), page. 993; 3-hydroxy-2-phenyl-4-quinoline carboxylic acid (R₂ is OH, CAS=[485-89-2]) is prepared in accordance with the methods described in U.S. Pat. No. 2,776,290 (1957); and 3-amino-2-phenyl-4-quinoline carboxylic (R₂ is NH₂, CAS=[36735-26-9]) is prepared in accordance with the methods described in Chemical Abstract 77:61769u (c.f. Khim. Geterotsikl. Soedin. (1972), 4 525-6).

Compounds of formula (III) and (V) are commercially available compounds (particularly when R′=alkyl) or they can be prepared from known compounds by known methods, for example, compounds of formula (III) in which R′ is alkoxycarbonyl and R′₄ is hydrogen and Ar′ is as defined for the compounds of formula (I), are described in Liebigs Ann. der Chemie, 523, 199, 1936.

The compounds of formula (IV) are known compounds or they are prepared using methods analogous to those used to prepare known compounds, for example those disclosed in in U.S. Pat. No. 4,386,091(Mead Johnson) and U.S. Pat. No. 4,487,773 (Mead Johnson).

It will be appreciated that in any of the above mentioned reactions any reactive group in the substrate molecule may be protected according to conventional chemical practice.

Suitable protecting groups in any of the above mentioned reactions are those used conventionally in the art. Thus, for example suitable hydroxyl protecting groups include benzyl or trialkylsilyl groups.

The methods of formation and removal of such protecting groups are those conventional methods appropriate to the molecule being protected. Thus for example a benzyloxy group may be prepared by treatment of the appropriate compound with a benzyl halide, such as benzyl bromide, and thereafter, if required, the benzyl group may be conveniently removed using catalytic hydrogenation or a mild ether cleavage reagent such as trimethylsilyl iodide or boron tribromide.

As indicated above, the compounds of formula (I) have useful pharmaceutical properties, accordingly the present invention also provides a compound of formula (I), or a pharmaceutically acceptable salt or solvate thereof, for use as an active therapeutic substance.

The present invention further provides a pharmaceutical composition comprising a compound of formula (I), or a pharmaceutically acceptable salt or solvate thereof, and a pharmaceutically acceptable carrier.

The present invention also provides the use of a compound of formula (I), or a pharmaceutically acceptable salt or solvate thereof, in the manufacture of a medicament for the treatment of the Primary and Secondary Conditions. Such a medicament, and a composition of this invention, may be prepared by admixture of a compound of the invention with an appropriate carrier. It may contain a diluent, binder, filler, disintegrant, flavouring agent, colouring agent, lubricant or preservative in conventional manner.

These conventional excipients may be employed for example as in the preparation of compositions of known agents for treating the conditions.

Preferably, a pharmaceutical composition of the invention is in unit dosage form and in a form adapted for use in the medical or veterinarial fields. For example, such preparations may be in a pack form accompanied by written or printed instructions for use as an agent in the treatment of the conditions.

The suitable dosage range for the compounds of the invention depends on the compound to be employed and on the condition of the patient. It will also depend, inter alia, upon the relation of potency to absorbability and the frequency and route of administration.

The compound or composition of the invention may be formulated for administration by any route, and is preferably in unit dosage form or in a form that a human patient may administer to himself in a single dosage. Advantageously, the composition is suitable for oral, rectal, topical, parenteral, intravenous or intramuscular administration. Preparations may be designed to give slow release of the active ingredient.

Compositions may, for example, be in the form of tablets, capsules, sachets, vials, powders, granules, lozenges, reconstitutable powders, or liquid preparations, for example solutions or suspensions, or suppositories.

The compositions, for example those suitable for oral administration, may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinyl-pyrrolidone, sodium starch glycollate or microcrystalline cellulose; or pharmaceutically acceptable setting agents such as sodium lauryl sulphate.

Solid compositions may be obtained by conventional methods of blending, filling, tabletting or the like. Repeated blending operations may be used to distribute the active agent throughout those compositions employing large quantities of fillers. When the composition is in the form of a tablet, powder, or lozenge, any carrier suitable for formulating solid pharmaceutical compositions may be used, examples being magnesium stearate, starch, glucose, lactose, sucrose, rice flour and chalk. Tablets may be coated according to methods well known in normal pharmaceutical practice, in particular with an enteric coating. The composition may also be in the form of an ingestible capsule, for example of gelatin containing the compound, if desired with a carrier or other excipients.

Compositions for oral administration as liquids may be in the form of, for example, emulsions, syrups, or elixirs, or may be presented as a dry product for reconstitution with water or other suitable vehicle before use. Such liquid compositions may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; aqueous or non-aqueous vehicles, which include edible oils. for example almond oil, fractionated coconut oil, oily esters. for example esters of glycerine, or propylene glycol, or ethyl alcohol, glycerine, water or normal saline; preservatives, for example methyl or propyl p-hydroxybenzoate or sorbic acid; and if desired conventional flavouring or colouring agents.

The compounds of this invention may also be administered by a non-oral route. In accordance with routine pharmaceutical procedure, the compositions may be formulated. for example for rectal administration as a suppository. They may also be formulated for presentation in an injectable form in an aqueous or non-aqueous solution, suspension or emulsion in a pharmaceutically acceptable liquid, e.g. sterile pyrogen-free water or a parenterally acceptable oil or a mixture of liquids. The liquid may contain bacteriostatic agents, anti-oxidants or other preservatives, buffers or solutes to render the solution isotonic with the blood, thickening agents, suspending agents or other pharmaceutically acceptable additives. Such forms will be presented in unit dose form such as ampoules or disposable injection devices or in multi-dose forms such as a bottle from which the appropriate dose may be withdrawn or a solid form or concentrate which can be used to prepare an injectable formulation.

The compounds of this invention may also be administered by inhalation, via the nasal or oral routes. Such administration can be carried out with a spray formulation comprising a compound of the invention and a suitable carrier, optionally suspended in, for example, a hydrocarbon propellant.

Preferred spray formulations comprise micronised compound particles in combination with a surfactant, solvent or a dispersing agent to prevent the sedimentation of suspended particles. Preferably, the compound particle size is from about 2 to 10 microns.

A further mode of administration of the compounds of the invention comprises transdermal delivery utilising a skin-patch formulation. A preferred formulation comprises a compound of the invention dispersed in a pressure sensitive adhesive which adheres to the skin, thereby permitting the compound to diffuse from the adhesive through the skin for delivery to the patient. For a constant rate of percutaneous absorption, pressure sensitive adhesives known in the art such as natural rubber or silicone can be used.

As mentioned above, the effective dose of compound depends on the particular compound employed, the condition of the patient and on the frequency and route of administration. A unit dose will generally contain from 20 to 1000 mg and preferably will contain from 30 to 500 mg, in particular 50, 100, 150, 200, 250, 300, 350, 400, 450, or 500 mg. The composition may be administered once or more times a day for example 2, 3 or 4 times daily, and the total daily dose for a 70 kg adult will normally be in the range 100 to 3000 mg. Alternatively the unit dose will contain from 2 to 20 mg of active ingredient and be administered in multiples, if desired, to give the preceding daily dose.

No unacceptable toxicological effects are expected with compounds of the invention when administered in accordance with the invention.

The present invention also provides a method for the treatment and/or prophylaxis of the Primary and Secondary Conditions in mammals, particularly humans, which comprises administering to the mammal in need of such treatment and/or prophylaxis an effective amount of a compound of formula (I) or a pharmaceutically acceptable salt or solvate thereof.

The activity of the compounds of the present invention, as NK₃ ligands, is determined by their ability to inhibit the binding of the radiolabelled NK₃ ligands. [¹²⁵I]-[Me-Phe⁷]-NKB or [³H]-Senktide, to guinea-pig and human NK₃ receptors (Renzetti et al. 1991, Neuropeptide, 18, 104-114; Buell et al, 1992, FEBS, 299(1), 90-95; Chung et al, 1994, Biochem. Biophys. Res. Commun., 198(3), 967-972).

The binding assays utilized allow the determination of the concentration of the individual compound required to reduce by 50% the [¹²⁵I]-[Me-Phe⁷]-NKB and [³H]-Senktide specific binding to NK₃ receptor in equilibrium conditions (IC₅₀).

Binding assays provide for each compound tested a mean IC₅₀ value of 2-5 separate experiments performed in duplicate or triplicate. The most potent compounds of the present invention show IC₅₀ values in the range 0.1-1000 nM. The NK3-antagonist activity of the compounds of the present invention is determined by their ability to inhibit senktide-induced contraction of the guinea-pig ileum (Maggi et al, 1990, Br. J. Pharmacol., 101, 996-1000) and rabbit isolated iris sphincter muscle (Hall et al., 1991, Eur. J. Pharmacol., 199, 9-14) and human NK₃ receptors-mediated Ca⁺⁺ mobilization (Mochizuki et al. 1994, J. Biol. Chem., 269, 9651-9658). Guinea-pig and rabbit in-vitro functional assays provide for each compound tested a mean K_{B} value of 3-8 separate experiments, where K_{B} is the concentration of the individual compound required to produce a 2-fold rightward shift in the concentration-response curve of senktide. Human receptor functional assay allows the determination of the concentration of the individual compound required to reduce by 50% (IC₅₀ values) the Ca⁺⁺ mobilization induced by the agonist NKB. In this assay, the compounds of the present invention behave as antagonists.

The therapeutic potential of the compounds of the present invention in treating the conditions can be assessed using rodent disease models.

As stated above, the compounds of formula (I) are also considered to be useful as diagnostic tool. Accordingly, the invention includes a compound of formula (I) for use as diagnostic tools for assessing the degree to which neurokinin-3 receptor activity (normal, overactivity or underactivity) is implicated in a patient's symptoms. Such use comprises the use of a compound of formula (I) as an antagonist of said activity, for example including but not restricted to tachykinin agonist-induced inositol phosphate turnover or electrophysiological activation, of a cell sample obtained from a patient. Comparison of such activity in the presence or absence of a compound of formula (I), will disclose the degree of NK-3 receptor involvement in the mediation of agonist effects in that tissue.

The following Descriptions illustrate the preparation of the intermediates, whereas the Examples illustrate the preparation of the compounds of the present invention. The compounds of the Examples are summarised in Tables 1-3 below.

Description 1

### 3-Morpholinomethyl-2-phenylquinoline-4-carboxylic Acid Hydrochloride

5.60 g (21.27 mmol) of 3-methyl-2-phenylquinoline-4-carboxylic acid (CAS [43071-45-0]) were dissolved in 100 ml of CH₂Cl₂; 7.60 g (42.50 mmol) of N-bromosuccinimide and 0.52 g (2.00 mmol) of dibenzoyl peroxide were added and the suspension was refluxed for 24 hours.

After cooling, the reaction mixture was evaporated in-vacuo to dryness, dissolved in 100 ml of THF and added to 50 ml (573.92 mmol) of morpholine. Then, it was stirred at room temperature overnight, evaporated in-vacuo to dryness and purified by gradient flash column chromatography on 230-400 mesh silica gel using a mixture of CH₂Cl₂/MeOH 95:5 containing 0.5% NH₄OH (28%) as starting eluent and a mixture of CH₂Cl₂/MeOH 80:20 containing 2% NH₄OH (28%) as final eluent. The product obtained was dissolved in acetone and acidified with HCl/Et₂O; the precipitate so formed was recovered by suction filtration; 0.85 g of the title compound were obtained as a white solid.

C₂₁H₂₀N₂O₃.HCl

M.P.=173-175° C.

M.W.=384.87

I.R. (Nujol): 3700-3100; 2750-2000; 1710; 1630 cm⁻¹.

Description 2

### (S)-N-(α-ethylbenzyl)-3-hydroxy-2-phenylquinoline-4-carboxamide

2.49 g (9.4 mmol) of 3-hydroxy-2-phenylquinoline-4-carboxylic acid (CAS [485-89-2]) were suspended in 150 ml of a 7/3 mixture of THF/CH₃CN; 1.40 g (10.3 mmol) of 1-hydroxybenzotriazole (HOBT) and 1.27 g (9.4 mmol) of (S)-α-ethylbenzylamine dissolved in 20 ml of CH₂Cl₂ were added and the reaction mixture was stirred at room temperature for 30 minutes. 2.13 g (10.3 mmol) of dicyclohexylcarbodiimide (DCC) dissolved in 20 ml of CH₂Cl₂ were added dropwise. The reaction was left at room temperature overnight, quenched with 20 ml of H₂O, evaporated in-vacuo to dryness and dissolved in EtOAc. The precipitated dicyclohexylurea was filtered off and the organic layer was washed with H₂O, 20% citric acid, sat. sol. NAHCO₃, sat. sol. NaCl. The organic layer was separated, dried over Na₂SO₄ and evaporated in-vacuo to dryness; the residue was purified by gradient column chromatography on 60-240 mesh silica gel using a mixture of hexane/EtOAc 9:1 as starting eluent and a mixture of hexane/EtOAc 7:3 as final eluent. The crude product was recrystallized from i-PrOH to yield 1.75 g of the title compound as a white solid.

C₂₅H₂₂N₂O₂

M.P.=168-168.4° C.

M.W.=382.47

[α]_{D}²⁰=−28.5 (c=0.5, MeOH)

Elemental analysis: Calcd. C, 78.51; H, 5.80; N, 7.33; Found C, 78.49; H, 5.84; N, 7.26.

I.R. (KBr): 3370; 1625; 1525 cm⁻¹.

300 MHz ¹H-NMR (DMSO-d₆): δ 9.80 (s, 1H); 9.11 (d, 1H); 8.00-7.94 (m, 3H); 7.61-7.42 (m, 8H); 7.38 (dd, 2H); 7.28 (dd, 1H); 5.06 (dt, 1H); 1.82 (ddq, 2H); 0.97 (t, 3H).

MS (EI; TSQ 700; source 200 C;70 V; 200 uA): 382 (M+.); 264; 247; 219.

Description 3

### (S)-N-(α-ethylbenzyl)-3-(ethoxycarbonylmethoxy)-2-phenylquinoline-4-carboxamide

2.0 g (5.2 mmol) of (S)-N-(α-ethylbenzyl)-3-hydroxy-2-phenylquinoline-4-carboxamide (compound of Description 2) were dissolved, under nitrogen atmosphere, in 20 ml of THF; 2.0 g (14.5 mmol) of K₂CO₃, 0.87 ml (7.8 mmol) of ethyl bromoacetate and a catalytic amount of KI were added and the mixture was stirred at room temperature for 2 hours and 30 minutes.

After filtering off the inorganic salts, the solution was evaporated in-vacuo to dryness, dissolved in EtOAc and washed with water; the organic layer was separated, dried over Na₂SO₄ and evaporated in-vacuo to dryness to obtain 3.3 g of a yellow oil.

This oil was purified by flash column chromatography on 230-400 mesh silica gel, eluting with a mixture of hexane/EtOAc 7:3 containing 0.5% NH₄OH (28%). The crude solid obtained was triturated with i-Pr₂O/i-PrOH, filtered. washed and dried to yield 2.1 g of the title compound as a white solid.

C₂₉H₂₈N₂O₄

M.P.=103-105° C.

M.W.=468.56

[α]^{D}²⁰=−42.5 (c=0.5, MeOH)

Elemental analysis: Calcd. C, 74.34; H, 6.02; N, 5.98; Found C, 74.44; H, 6.01; N, 6.00.

I.R. (KBr): 3320-3140; 3100-3020; 2980-2920; 1758; 1630; 1550 cm⁻¹.

300 MHz ¹H-NMR (DMSO-d₆): δ 9.28 (d, 1H); 8.08 (d, 1H); 8.05-7.98 (m, 2H); 7.80-7.71 (m. 1H): 7.60 (d, 2H); 7.55-7.48 (m, 3H); 7.43 (d, 2H); 7.35 (dd, 2H); 7.28 (dd, 1H); 5.06 (dt, 1H); 4.26 (ABq, 2H); 4.04 (q, 2H); 1.86-1.67 (mn, 2H); 1.12 (t, 3H); 0.96 (t, 3H).

MS (EI; TSQ 700; source 180 C;70 V; 200 uA): 468 (M+.); 439; 334; 306; 278.

Description 4

### (S)-N-(α-ethylbenzyl)-2-phenyl-3-(2-phthalimidoethoxy)quinoline-4-carboxamide

1.90 g (5.0 mmol) of (S)-N-(α-ethylbenzyl)-3-hydroxy-2-phenylquinoline-4-carboxamide (product of Description 2) were dissolved in 20 ml of THF.

3.80 g (14.9 mmol) of N-(2-bromoethyl)phthalimide dissolved in 15 ml of THF, 2.00 g (14.5 mmol) of K₂CO₃ and 0.25 g of KI were added and the suspension was stirred at room temperature for 2.5 hours and then refluxed for 2 hours.

Additional 1.90 g (7.4 mmol) of N-(2-bromoethyl)phthalimide and a catalytic amount of KI were added and the reaction refluxed for 3.5 hours; additional 0.50 g (2.0 mmol) of N-(2-bromoethyl)phthalimide and a catalytic amount of KI were added and the reaction refluxed for 5 hours.

The inorganic salts were filtered off and the reaction mixture evaporated in-vacuo to dryness, dissolved in CH₂Cl₂ and washed with water; the organic layer was separated, dried over Na₂SO₄ and evaporated in-vacuo to dryness. The residue was purified by flash column chromatography on 230-400 mesh silica gel, eluting initially with a mixture of hexane/ethyl acetate 8:2 containing 0.5% NH₄OH (28%) and then with a mixture of hexane/ethyl acetate 3:2 containing 0.5% NHAOH (28%). The crude solid obtained (2.60 g) was triturated with i-Pr₂O, filtered, washed and dried to yield 2.5 g of the title compound.

C₃₅H₂₉N₃O₄

M.P.=172-175° C.

M.W.=555.64

[α]_{D}²⁰=−16.3 (c=0.5, MeOH)

I.R. (KBr): 3280; 3060; 2960; 1780; 1715; 1660; 1530 cm⁻¹.

300 MHz ¹H-NMR (DMSO-d₆): δ 9.27 (d, 1H); 8.03 (d, 1H); 7.92-7.84 (m, 4H); 7.78-7.69 (m, 3H); 7.60-7.53 (m, 2H); 7.46-7.38 (m, 4H); 7.27 (dd, 1H); 7.13-7.04 (m, 3H); 4.96 (dt, 1H); 3.92-3.78 (mn, 2H); 3.72-3.55 (m, 2H); 1.78 (dq, 2H); 0.93 (t, 3H).

MS (EI; TSQ 700; source 180 C;70 V; 200 uA): 555 (M+.), 526,421, 174.

Description 5

### (S)-N-(α-ethylbenzyl)-3-(2-aminoethoxy)-2-phenylquinoline-4-carboxamide

2.2 g (3.9 mmol) of (S)-N-(α-ethylbenzyl)-2-phenyl-3-(2-phthalimidoethoxy) quinoline-4-carboxamide (compound of Description 4) were dissolved in 150 ml of 96% EtOH; the solution was heated to reflux; 0.38 ml (7.8 mmol) of hydrazine hydrate were added and the reaction mixture refluxed for 4 hours.

Additional 0.4 ml (8.2 mmol), 0.2 ml (4.1 mmol), 0.2 ml (4.1 mmol), 0.4 ml (8.2 mmol), 0.4 ml (8.2 mmol) of hydrazine hydrate were added every 12 hours while refluxing the reaction mixture. Then it was evaporated in-vacuo to dryness and 20 ml of H₂0 were added; it was cooled with an ice bath and 10 ml of conc. HCl were added.

The reaction mixture was refluxed for 1 hour and then, after cooling, the phthalhydrazide was filtered off. The resulting aqueous filtrate was washed with EtOAc, basified with 2N NaOH and extracted with EtOAc. The organic layer was washed with sat. sol. NaCl, dried over Na₂SO₄ and evaporated in-vacuo to dryness. The residue was purified by flash column chromatography on 230-400 mesh silica gel, eluting with a mixture of EtOAc/MeOH 96:4 containing 1.2% NH₄OH (28%) to yield 1.2 g of the title compound.

C₂₇H₂₇N₃O₂

M.P.=62-66° C.

M.W.=425.54

I.R. (KBr):.3360; 3250; 3060: 3020; 2960; 2920; 2870; 1640; 1540 cm⁻¹.

300 MHz ¹H-NMR (DMSO-d₆): δ 9.45 (d, 1H); 8.09 (d, 1H); 8.00 (dd. 1H); 7.94 (s br, 3H); 7.76 (ddd, 1H); 7.65-7.51 (m. 4H); 7.48-7.40 (m, 3H); 7.31 (dd, 1H); 5.09 (dt, 1H); 3.83 (t, 2H); 2.72 (m, 2H); 1.93-1.80 (m, 2H); 0.99 (t, 3H).

MS (FAB POS; thioglycerine matrix; FAB gas Xe; 8 kV; source 50): 426 (MH+).

Description 6

### (S)-N-(α-ethylbenzyl)-3-formylmethoxy-2-phenylquinoline-4-carboxamide

0.64 ml (7.4 mmol) of oxalyl chloride were dissolved, under nitrogen atmosphere, in 5 ml of dry CH₂Cl₂. The solution was cooled at −55° C. and 0.53 ml (7.4 mmol) of DMSO dissolved in 1.5 ml of dry CH₂Cl₂ were added dropwise, keeping the temperature at −55° C. The solution was maintained under stirring for 7 minutes, then 2.1 g (4.9 mmol) of (S)-N-(α-ethylbenzyl)-3-(2-hvdroxyethoxy)-2-phenylquinoline-4-carboxamide (compound of Example 2) dissolved in 50 ml of dry CH₂Cl₂ were added dropwise, maintaining the temperature between −55 and −50° C. After 30 minutes 4.6 ml (33.0 mmol) of TEA were added dropwise and the temperature was allowed to raise to room temperature. 10 ml of H₂O were added, the organic layer was separated and washed with H₂O, 20% citric acid, sat. sol. NaHCO₃, sat. sol. NaCl, dried over Na₂SO₄, filtered and evaporated in-vacuo to dryness.

The residue was purified by gradient flash column chromatography on 230-400 mesh silica gel using as starting eluent a mixture of hexane/EtOAc 70:30 containing 0.5% NH₄OH (28%) and as final eluent EtOAc containing 0.5% NH₄OH (28%). The crude product was triturated with i-Pr₂O to yield 0.53 g of the title compound, used without further purification.

C₂₇H₂₄N₂O₃

M.W.=424.50

### EXAMPLE 1

### (S)-N(α-ethylbenzyl)-3-morpholinomethyl-2-phenylquinoline-4-carboxamide

0.8 g (2.1 mmol) of 3-morpholinomethyl-2-phenylquinoline-4-carboxylic acid hydrochloride (compound of Description 1) were dissolved, under nitrogen atmosphere, in 25 ml of a 8:2 mixture of THF/CH₃CN; after cooling at −10° C., 0.31 g (2.3 mmol) of 1-hydroxybenzotriazole (HOBT), 0.29 ml (2.9 mmol) of TEA and 0.34 g (2.5 mmol) of (S)-α-ethylbenzylamine were added. The reaction mixture was stirred for 5 minutes at a temperature between −10 and −5° C., then 0.47 g (2.3 mmol) of dicyclohexylcarbodiimide (DCC) were added.

The temperature was allowed to raise to room temperature and the reaction was maintained under stirring for 6 hours and on standing overnight; then it was evaporated in-vacuo to dryness, dissolved in CH₂Cl₂, and washed with sat. sol. NaHCO₃. The organic layer was evaporated in-vacuo to dryness, dissolved in 1N HCl, washed with i-Pr₂O, basified with sat. sol. NaHCO₃ and extracted with CH₂Cl₂. The solvent was evaporated in-vacuo to dryness and the residue was chromatographed on 60-240 mesh silica gel, eluting with a mixture of hexane/EtOAc 7:3 containing 1% NH₄OH (28%) first and then with a mixture of hexane/EtOAc 3:2 containing 1% NH₄OH (28%).

The crude product was dissolved in acetone and the solution acidified with HCl/Et₂O; the solid was recovered by suction filtration and triturated with warm toluene to yield 0.43 g of the title compound as a pale yellow solid.

C₃₀H₃₁N₃O₂.HCl

M.P.=173-176° C.

M.W.=502.06

[α]_{D}²⁰=+11.0 (c=0.5, MeOH)

I.R (Nujol): 3600-3300; 3150; 2750-2020; 1655; 1630; 1545 cm⁻¹.

300 MHz ¹H-NMR (DMSO-d₆): δ 9.42 (d br, 1H); 8.09 (d, 1H); 7.85 (ddd, 1H); 7.79 (d br, 1H); 7.66-7.11 (m, 11H); 5.04 (dt, 1H); 4.05 (s br, 2H); 3.46 (t, 4H); 2.50-2.30 (m, 4H); 2.10-1.84 (m, 2H); 0.99 (t, 3H).

MS (EI; TSQ 700; source 180 C;70 V; 200 uA): 465 (M+.); 380; 330; 261; 217.

### EXAMPLE 2

### (S)-N-(α-ethylbenzyl)-3-(2-hydroxyethoxy)-2-phenylquinoline-4-carboxamide

0.65 g (1.4 mmol) of (S)-N-(α-ethylbenzyl)-3-(ethoxycarbonylmethoxy)-2-phenyl quinoline-4-carboxamide (compound of Description 3) were dissolved, under nitrogen atmosphere, in 50 ml of t-BuOH; 55 mg (1.4 mmol) of NaBH₄ were added and the mixture was heated to reflux. 7 ml of MeOH were added dropwise, the reaction was refluxed for 3 hours and then quenched with 5 ml of sat. sol. NH₄Cl, evaporated in-vacuo to dryness, dissolved with CH₂Cl₂ and washed with sat. sol. NaCl. The extracted organic layer was dried over Na₂SO₄, filtered and evaporated in-vacuo to dryness to yield 0.75 g of a crude product which was purified by gradient flash column chromatography on 230-400 mesh silica gel using a mixture of hexane/EtOAc 80:20 containing 0.5% NH₄OH (28%) as starting eluent and a mixture of hexane/EtOAc 50:50 containing 0.5% NH₄OH (28%) as final eluent. The purified product obtained was triturated with warm i-PrOH to yield 0.28 g of the title compound as a white solid.

C₂₇H₂₆N₂O₃

M.P.=129-130° C.

M.W.=426.52

[α]_{D}²⁰=−41.2 (c=0.5, MeOH)

Elemental Analysis: Calcd. C, 76.03; H, 6.14: N, 6.57; Found C, 76.02; H, 6.17; N, 6.58.

I.R. (KBr): 3240; 3060; 2980-2920; 1625; 1550 cm⁻¹.

300 MHz ¹H-NMR (DMSO-d₆): δ 9.30 (d, 1H); 8.07-7.90 (m, 3H); 7.76-7.67 (m, 1H);

7.60-7.49 (m, 5H); 7.45 (d, 2H); 7.39 (dd, 2H); 7.29 (dd, 1H); 5.08 (dt, 1H); 4.57 (t, 1H); 3.69 (m, 2H); 3.34 (dt, 2H); 1.82 (m, 2H); 0.99 (t, 3H).

MS (EI; TSQ 700; source 180 C;70 V; 200 uA): 426 (M+); 397; 292; 264

### EXAMPLE 3

### (S)-N-(α-ethylbenzyl)-3-hydroxy-7-methyl-2-phenylquinoline-4-carboxamide

0.5 g (1.8 mmol) of 3-hydroxy-7-methyl-2-phenylquinoline-4-carboxylic acid were dissolved, under nitrogen atmosphere, in 35 ml of dry THF and 20 ml of CH₃CN. 0.25 g (1.8 mmol) of (S)-α-ethylbenzylamine and 0.45 g (3.4 mmol) of HOBT were added; the solution was cooled at 0° C. and 0.41 g (2.0 mmol) of DCC, dissolved in 12 ml of dry CH₂Cl₂, were added dropwise. The mixture was stirred 1 hour at 0° C., 2 hours at room temperature and 2 hours at 40° C.; after cooling the precipitated dicyclohexylurea was filtered off and the filtrate was evaporated in-vacuo to dryness. The residue was dissolved in CH₂Cl₂ and washed with 20% citric acid, sat. sol. NaHCO₃ and sat. sol. NaCl; the organic layer was dried over Na₂SO₄, filtered and evaporated in-vacuo to dryness. The crude product was purified by flash column chromatography on 230400 mesh silica gel eluting with CH₂Cl₂ containing 0.5% NH₄OH (28%); the product was further purified by preparative HPLC to yield 30 mg of the title compound as a white solid.

C₂₆H₂₄N₂O₂

M.P.=111-114° C.

M.W.=396.48

I.R. (KBr): 3310; 3100-3020; 2980-2820; 1625; 1578; 1555; 1540 cm⁻¹.

300 MHz ¹H-NMR (DMSO-d₆): δ 9.60 (s br, 1H); 9.02 (s br, 1H); 7.96 (d br, 2H); 7.76 (s br, 1H); 7.54-7.24 (m, 10H); 5.05 (dt, 1H); 2.47 (s, 3H); 1.80 (m, 2H); 0.95 (t, 3H).

MS (EI; TSQ 700; source 180 C;70 V; 200 uA): 396 (M+); 367; 278; 261; 233.

### EXAMPLE 4

### (S)-N-(α-ethylbenzyl)-3-fluoro-2-phenylquinoline-4-carboxamide

0.54 g (4.0 mmol) of (S)-α-ethylbenzylamine and 0.7 ml (5.0 mmol) of TEA were dissolved, under nitrogen atmosphere, in 10 ml of dry CH₂Cl₂; 1.14 g (4.0 mmol) of 3-fluoro-2-phenylquinoline-4-carbonylchloride (obtained from the corresponding carboxylic acid by reaction with oxalyl chloride in CH₂Cl₂ at room temperature), dissolved in 20 ml of a 1:1 mixture of dry CH₂Cl₂/DMF, were added dropwise and the reaction was maintained at room temperature overnight

The reaction mixture was evaporated in-vacuo to dryness and the residue dissolved in EtOAc and washed with H₂O, 5% citric acid, sat. sol. NaHCO₃ and sat. sol. NaCl. The organic layer was dried over Na₂SO₄, filtered and evaporated in-vacuo to dryness. The residual oil was purified by gradient flash column chromatography on 230-400 mesh silica gel using hexane as starting eluent and a mixture of hexane/EtOAc 9:1 as final eluent to yield 0.5 g of the title compound.

C₂₅H₂₁FN₂O

M.P.=67-68° C.

M.W.=384.46

[α]_{D}²⁰=−22.8 (c=0.5, MeOH)

I.R. (KBr): 3250; 3060; 2960; 2930; 1640; 1600; 1550 cm⁻¹.

300 MHz ¹H-NMR (DMSO-d₆): δ 9.50 (d, 1H); 8.17 (d, 1H); 8.01 (m, 2H); 7.81 (dd, 1H); 7.76-7.66 (m, 2H); 7.64-7.56 (m, 3H); 7.46-7.35 (m, 4H); 7.29 (dd, 1H); 5.10 (dt, 1H); 1.88-1.74 (m, 2H); 0.99 (t, 3H).

MS (EI; TSQ 700; source 180 C:70 V; 200 uA ): 384 (M+); 355; 250; 222.

### EXAMPLE 5

### (S)-N-(α-ethylbenzyl)-3-[2-(2-isoindolinyl)ethoxy]-2-phenylquinoline-4-carboxamide Dihydrochloride

1.5 g (3.5 mmol) of (S)-N-(α-ethylbenzyl)-3-(2-aminoethoxy)-2-phenylquinoline-4-carboxamide (compound of Description 5) and 1.0 g (3.9 mmol) of α,α′-dibromo-o-xylene were dissolved in 150 ml of DMF; 1.1 ml (7.8 mmol) of TEA and a catalytic amount of KI were added and the mixture was heated to 80° C. for 3 hours. The reaction mixture was evaporated in-vacuo to dryness, dissolved in 10% HCl and washed with hexane. Then it was basified with 20% NaOH and extracted with CH₂Cl₂; the organic layer was washed with sat. sol. NaCl, dried over Na₂SO₄, filtered and evaporated in-vacuo to dryness. The residue was purified by flash column chromatography on 230-400 mesh silica gel eluting with a mixture of hexane/EtOAc 7:3 containing 0.5% NH₄OH (28%); the product was further purified by preparative HPLC, dissolved in EtOAc and the solution acidified with HCl/Et₂O to yield 100 mg of the title compound as a gray solid.

C₃₅H₃₃N₃O₂.2HCl

M.P.=95° C. dec.

M.W.=600.59

I.R. (KBr): 3700-3100; 3080-3020; 2980-2820; 2740-2020; 1650; 1550 cm⁻¹.

300 MHz ¹H-NMR (DMSO-d₆): δ 11.38 (s br, 1H); 9.49 (d, 1H); 8.10 (d, 1H); 7.95 (m, 2H); 7.78 (ddd, 1H); 7.67-7.55 (m, 5H); 7.48-7.22 (m, 9H); 5.06 (dt, 1H); 4.50-3.50 (m, 2H); 4.30-4.12 (m, 2H); 4.12-3.97 (mn, 2H); 3.28 (m. 2H); 1.98-1.72 (m, 2H); 0.94 (t, 3H).

MS (EI; TSQ 700: source 180 C;70 V: 200 uA): 527 (M): 525; 383; 249.

### EXAMPLE 6

### (S)-N-(α-ethylbenzyl)-3-(2-homophthalimidoethoxy)-2-phenylquinoline-4-carboxamide

0.95 g (2.2 mmol) of the compound of Description 5 and 0.47 g (2.9 mmol) of omophthalic anhydride were dissolved in 20 ml of toluene; some triturated molecular sieves were added and the solution was refluxed, under magnetic stirring, distilling away the forming H₂O with a Dean-Stark apparatus.

The reaction was refluxed for 13 hours then, after cooling, the molecular sieves were filtered off and it was evaporated in-vacuo to dryness. The residue was dissolved in CH₂Cl₂) and washed with H₂O, 20% citric acid, sat. sol. NaHCO₃ and sat. sol. NaCl; the organic layer was dried over Na₂SO₄, filtered and evaporated in-vacuo to dryness. The crude product was purified by gradient flash column chromatography on 230400 mesh silica gel using a mixture of hexane/EtOAc 70:30 containing 0.5% NH₄OH (28%) as starting eluent and a mixture of hexane/EtOAc 50:50 containing 0.5% NH₄OH (28%) as final eluent. The crude product was triturated with warm i-Pr₂O/i-PrOH to yield 0.55 g of the title compound as a white solid.

C₃₆H₃₁N₃O₄

M.P.=159-161° C.

M.W.=569.67

[α]_{D}²⁰=−29.7 (c=0.5, MeOH)

Elemental analysis: Calcd. C, 75.90; H, 5.48; N, 7.38; Found C, 75.73; H, 5.45; N, 7.36.

I.R. (KBr): 3360; 3100-3020; 2980-2820; 1715; 1668; 1610; 1510 cm⁻¹.

300 MHz ¹H-NMR (DMSO-d₆): δ 9.25 (d, 1H); 8.05 (d, 1H); 8.00 (d, 1H); 7.79 (m, 2H); 7.71 (m, 2H); 7.58-7.35 (m, 8H); 7.27-7.23 (m, 4H); 4.98 (dt, 1H); 4.09-3.79 (m, 6H); 1.79 (m, 2H); 0.93 (t, 3H).

MS (EI; TSQ 700; source 180 C;10 V; 200 uA): 569 (M+); 382; 187.

### EXAMPLE 7

### (S)-N-(α-ethylbenzyl)-2-phenyl-[2-(1,2,3,4-tetrahydro-2-isoquinolinyl)ethoxy]quinoline-4-carboxamide Hydrochloride

0.5 g (1.2 mmol) of (S)-N-(α-ethylbenzyl)-3-formylmethoxy-2-phenylquinoline-4-carboxamide (compound of Description 6) and 0.3 ml (2.4 mmol) of 1,2.3,4-tetrahydroisoquinoline were dissolved, under nitrogen atmosphere, in 10 ml of CH₃CN: Some triturated molecular sieves were added and the solution was maintained under stirring at room temperature for 30 minutes; 0.2 g (3.2 mmol) of NaCNBH₃ were then added in 30 minutes. The reaction mixture was maintained at room temperature overnight, then was quenched with 15% NaOH, keep under stirring for 20 minutes and evaporated ini-vacuo to dryness. The residue was dissolved in 10% HCl, washed with Et₂O, basified with 15% NaOH and extracted with Et₂O. The organic layer was washed with H₂O, dried over Na₂SO₄, filtered and evaporated in-vacuo to dryness. The residue was purified by flash column chromatography on 230-400 mesh silica gel eluting with a mixture of hexane/EtOAc 7:3 containing 0.5% NH₄OH (28%) to obtain 140 mg of a product which was dissolved in MeOH and acidified with HCl/Et₂O. The solvent was evaporated in-vacuo to dryness and the residue was triturated with warm i-Pr₂O/i-PrOH to yield 120 mg of the title compound.

C₃₆H₃₅N₃O₂.HCl

M.P.=120-130° C. dec.

M.W.=578.16

[α]_{D}²⁰=−14.8 (c=0.5, MeOH)

I.R. (KBr): 3700-3100; 3080-3000; 2980-2820; 2800-2020; 1670-1640; 1550 cm⁻¹.

300 MHz ¹H-NMR (DMSO-d₆): δ 10.89 (s br. 1H); 9.60 (d, 1H); 8.09 (d, 1H); 7.95 (m, 2H); 7.78 (ddd, 1H); 7.65-7.52 (m, 5H); 7.44-7.22 (m, 8H); 7.08 (d br, 1H); 4.30-4.00 (m, 4H); 3.50-2.90 (m, 6H); 1.80 (m, 2H); 0.90 (m, 3H).

MS (EI; TSQ 700; source 180 C;70 V; 200 uA): 541 (M+); 383; 247; 159; 146; 132.

Description 7

### (R,S)-N-[α-(1-hydroxyethyl)benzyl]-3-hydroxy-2-phenylquinoline-4-carboxamide

Prepared as described in Description 2 from 0.98 g (3.7 mmol) of 3-hydroxy-2-phenylquinoline-4-carboxylic acid (CAS [485-89-2]), 1.5 g (3.9 mmol) of 1-amino-1-phenyl-2-propanol (diastereomeric mixture) (Viscontini, M., 1961, Helvetica Chimica Acta, 71, 631), 0.95 g (7.1 mmol) of HOBT, 0.51 ml (4.6 mmol) of N-methylmorpholine and 0.84 g (4.1 mmol) of DCC in 50 ml of a 2:1 mixture of THF and CH₃CN.

The work-up of the reaction mixture was carried out in the same manner as described in Description 2. The residual oil was purified by flash column chromatography on 230-400 mesh silica gel eluting with a mixture of EtOAc/MeOH 98:2 containing 0.5% NH₄OH (28%) to obtain a crude product which was triturated with i-PrOH to yield 690 mg of the title compound.

C₂₅H₂2N₂O₃

M.W.=398.46

I.R. (KBr): 3410; 3320; 3100-3000; 1635; 1580 cm⁻¹.

300 MHz ¹H-NMR (DMSO-d₆): δ 9.70 (s br, 1H); 9.15 (s br, 1H); 7.99 (d, 1H); 7.98 (dd, 2H); 7.67 (m, 1H); 7.59-7.42 (m, 7H); 7.35 (dd, 2H); 7.28 (dd, 1H); 5.16 (dd, 1H); 4.99 (s br, 1H); 4.02 (dq, 1H); 1.10 (d, 3H).

MS (EI; TSQ 700; source 180 C;70 V; 200 uA): 398 (M+); 354; 248; 106.

Description 8

### (S)-N-(α-ethylbenzyl)-3-[2-(2′-hydroxymethylphenylacetyl)aminoethoxy]-2-phenylquinoline-4-carboxamide

0.7 g (4.7 mmol) of isochromanone were dissolved in 25 ml of abs. EtOH: 2.0 g (4.7 mmol) of (S)-N-(α-ethylbenzyl)-3-(2-aminoethoxy)-2-phenylquinoline-4-carboxamide (compound of Description 5) were added and the reaction was refluxed for 12 hours. Additional 0.3 g (2.0 mmol) of isochromanone were added and the reaction mixture was refluxed for 5 hours; additional 0.5 g (3.4 mmol) of isochromanone were added and the reaction refluxed for 10 hours. After cooling, it was evaporated in vacuo to dryness and the residue was purified by gradient flash column chromatography on 230-400 mesh silica utilising a mixture of hexane/EtOAc 50:50 containing 0.5% NH₄OH (28%) as starting eluent and a mixture of hexane/EtOAc 20:80 containing 0.5% NH₄OH (28%) as final eluent. The crude product so obtained was triturated with i-Pr₂O/i-PrOH to yield 1.8 g of the title compound.

C₃₆H₃₅N₃O₄

M.P.=160-163° C.

M.W.=573.69

[α]_{D}²⁰=−31.5 (c=0.5, MeOH)

Elemental analysis: Calcd. C, 75.36; H, 6.15; N, 7.32; Found C, 75.09; H, 6.14; N, 7.34.

I.R. (KBr): 3600-3100; 3100-3000; 1641; 1558 cm⁻¹.

300 MHz ¹H-NMR (DMSO-d₆): δ 9.30 (d, 1H); 8.08 (d, 1H); 7.98 (m, 2H); 7.89 (t br, 1H); 7.73 (ddd, 1H); 7.59 (m, 2H); 7.57-7.48 (mn, 3H); 7.45 (m, 2H); 7.41-7.33 (m, 3H); 7.28 (dd. 1H); 7.19 (dd, 1H); 7.15 (dd, 1H); 7.09 (dd, 1H); 5.09 (t, 1H); 5.08 (dt, 1H); 4.48 (d, 1H); 3.70-3.59 (m, 2H); 3.37 (s, 2H); 3.12-2.92 (m, 2H); 1.90-1.75 (m, 2H); 0.99 (t, 3H).

MS (EI; TSQ 700; source 180 C;70 V; 200 uA): 555; 438; 411; 382; 247; 218; 192; 174; 119.

Description 9

### (S)-N-(α-ethylbenzyl)-3-[2-(3-carboxypropanoyl)aminoethoxy]-2-phenylquinoline-4-carboxamide

2.0 g (4.7 mmol) of (S)-N-(α-ethylbenzyl)-3-(2-aminoethoxy)-2-phenylquinoline-4-carboxamide (compound of Description 5) and 0.6 g (6.2 mmol) of succinic anhydride were dissolved in 50 ml of toluene; some triturated molecular sieves were added and the reaction mixture was refluxed in a Dean Stark apparatus for 4 hours. The reaction mixture was evaporated in vacuo to dryness, dissolved in 100 ml of CH₂Cl₂ and washed with sat. sol. NaCl, 20% citric acid and sat. sol. NaCl. The organic layer was dried over Na₂SO₄ and evaporated in vacuo to dryness to yield 2.3 g of the crude product which was purified by flash column chromatography on 230-400 mesh silica gel, eluting initially with a mixture CH₂Cl₂/MeOH 9:1 and then with a mixture of CH₂Cl₂/MeOH 8:2. The crude solid obtained was triturated with i-Pr₂O/i-PrOH, filtered, washed and dried to yield 1.4 g of the title compound.

C₃₁H₃₁N₃O₅

M.P. 118-122° C.

M.W.=525.60

[α]_{D}²⁰=−32.1 (c=0.5, MeOH)

I.R. (KBr): 3600-3120; 3100-3000: 1740-1700; 1680-1600 cm⁻¹.

300 MHz ¹H-NMR (DMSO-d₆): δ 11.98 (s br, 1H); 9.28 (d, 1H); 8.07 (d, 1H); 7.99 (dd, 2H); 7.73 (ddd, 1H); 7.66 (t br, 1H); 7.61-7.48 (m, 5H); 7.46 (d, 2H); 7.39 (dd, 2H); 7.30 (dd. 1H); 5.05 (dt, 1H); 3.69-3.57 (m, 2H); 3.12-2.91 (m, 2H); 2.34 (m, 2H); 2.21 (m, 2H); 1.90-1.75 (m. 2H); 1.00 (t, 3H).

MS (FAB POS; thioglycerine matrix; FAB gas Xe; 8 kV; source 50): 526 (MH); 383; 291.

Description 10

### (S,Z)-N-(α-ethylbenzyl)-3-[2-(3-carboxypropenoyl)aminoethoxy]-2-phenylquinoline-4-carboxamide

2.0 g (4.7 mmol) of (S)-N-(α-ethylbenzyl)-3-(2-aminoethoxy)-2-phenylquinoline-4-carboxamide (compound of Description 5) and 0.61 g (6.2 mmol) of maleic anhydride were dissolved in 50 ml of toluene. Some molecular sieves were added and the reaction mixture was refluxed for 5 hours. After cooling, the reaction mixture was evaporated in vacuo to dryness. dissolved in CH₂Cl₂ and washed with sat. sol. NaCl, 20% citric acid. sat. sol. NaCl. The organic layer was dried over Na₂SO₄ and evaporated in vacuo to dryness. The crude product was purified by flash column chromatography on 230-400 mesh silica gel, eluting with a mixture of i-Pr₂O/EtOAc 70:30 containing 0.5% of 85% formic acid, and then triturated with i-Pr₂O to yield 2.0 g of the title compound.

C_{3 1}H₂₉N₃O₅

M.P.=158-162° C.

M.W.=523.59

[α]_{D}²⁰=−38.6 (c=0.5. MeOH)

Elemental analysis: Calcd. C_{71.11;} H, 5.58; N,8.03; Found C, 70.90; H, 5.56; N, 7.95.

I.R (KBr): 3280; 3150-3000; 1710; 1640-1620 cm⁻¹.

300 MHz ¹H-NMR (DMSO-d₆): δ 14.80 (s br, 1H); 9.30 (d, 1H); 9.08 (t br, 1H); 8.07 (d, 1H); 7.94 (dd, 2H); 7.79-7.70 (m, 1H); 7.60 (m, 2H); 7.52-7.38 (m, 7H); 7.29 (dd, 1H); 6.32 (d. 1H); 6.27 (d, 1H); 5.07 (dt, 1H); 3.76-3.64 (m, 2H); 3.28-3.00 (m, 2H); 1.90-1.74 (m, 2H); 1.00 (t, 3H).

MS (EI; TSQ 700; source 180 C;70 V; 200 uA): 425; 407.

Description 11

### (S)-N-(α-ethylbenzyl)-3-(2-aminoacetylaminoethoxy)-2-phenylquinoline-4-carboxamide

3.0 g (7.1 mmol) of (S)-N-(α-ethylbenzyl)-3-(2-aminoethoxy)-2-phenylquinoline-4-carboxamide (compound of Description 5) were dissolved, under nitrogen atmosphere, in 60 ml of CH₂Cl₂. 1.2 ml (8.5 mmol) of TEA were added; the solution was cooled to 0° C. and 2.7 g (8.5 mmol) of (9-fluorenylmethoxycarbonyl)glycinyl chloride (FMOC-glycinyl chloride), dissolved in 60 ml of CH₂Cl₂, were added dropwise. The reaction mixture was stirred at room temperature for 3 hours and then washed with sat. sol. NaCl, 20% citric acid, sat. sol. NaHCO₃, sat. sol. NaCl, dried over Na₂SO₄ and evaporated in vacuo to dryness. The crude product was purified by gradient flash column chromatography on 230-400 mesh silica gel, utilising a mixture of hexan/EtOAc 1:1 as starting eluent and a mixture of EtOAc/MeOH 9:1 as final eluent. The product (5.0 g) was dissolved in 100 ml of a 10% solution of diethylamine in DMF and stirred at room temperature for 30 minutes. The reaction mixture was then evaporated in vacuo and purified by gradient flash column chromatography on 230-400 mesh silica gel, utilising a mixture of EtOAc/MeOH 9:1 as starting eluent and a mixture of EtOAc/MeOH 7:3 as final eluent, to yield 0.6 g of the title compound.

C₂₉H₃₀N₄O₃

M.P.=55-60° C. dec.

M.W.=482.58

[α]_{D}²⁰=−33.7 (c=0.5, MeOH)

Elemental analysis: Calcd. C, 72.12; H, 6.27; N. 11.61; Found C, 70.12; H, 6.45; N. 10.81.

I.R. (KBr): 3500-3110; 3100-3000; 1680-1650; 1638 cm⁻¹.

300 MHz ¹H-NMR (DMSO-d₆): δ 9.29 (d, 1H); 8.06 (d, 1H); 7.98 (dd, 2H); 7.74 (ddd, 1H); 7.68 (t br. 1H); 7.60-7.38 (in, 9H); 7.30 (dd, 1H); 5.09 (dt, 1H); 3.70-3.55 (m, 2H); 3.18-3.00 (m, 2H); 2.99 (s, 2H); 1.90-1.78 (m, 2H); 1.00 (t, 3H).

MS (EI; TSQ 700; source 180 C;70 V; 200 uA): 482 (M+); 382; 291; 264; 247; 219; 190; 141; 119; 101; 91.

Description 12

### (S)-N-(α-ethylbenzyl)-3-[2-(S)-α-aminophenylacetylaminoethoxy]-2-phenylquinoline-4-carboxamide

The reaction to obtain the FMOC-protected title compound was conducted as described in Description 11, starting from 2.8 g (6.7 mmol) of (S)-N-(α-ethylbenzyl)-3-(2-aminoethoxy)-2-phenylquinoline-4-carboxamide (compound of Description 5), 1.1 ml (8.0 mmol) of TEA and 3.1 g (8.0 mmol) of (S)-FMOC-phenylglycinyl chloride. The reaction was stirred at room temperature for 20 hours and worked up as described in Description 11 to yield 4.5 g of the FMOC protected title compound, which was deprotected by stirring at room temperature for 30 minutes with 90 ml of a 10% solution of diethylamine in DMF. The reaction mixture was then evaporated in vacuo and purified by gradient flash column chromatography on 230-400 mesh silica gel, utilising EtOAc as starting eluent and a mixture of EtOAc/MeOH 9:1 as final eluent, to yield, after trituration with i-Pr₂O, 1.4 g of the title compound.

C₃₅H₃₄N₄O₃

M.P.=140-145° C.

M.W.=558.68

[α]_{D}²⁰=−17.0 (c=0.5, MeOH)

Elemental analysis: Calcd. C, 75.25; H, 6.13; N, 10.03; Found C, 72.70; H, 6.11; N, 9.80.

I.R. (KBr): 3440-3110; 3100-3000; 1650-1630; 1585 cm⁻¹.

300 MHz ¹H-NMR (DMSO-d₆): δ 9.30 (d, 1H); 8.08 (d, 1H); 7.97 (dd, 2H); 7.92 (t br, 1H); 7.72 (dd, 1H); 7.60-7.48 (m, 5H); 7.45 (d, 2H); 7.38 (dd, 2H); 7.30-7.20 (m, 6H); 5.09 (dt, 1H); 4.27 (s, 1H); 3.65 (t, 2H); 3.07 (dt, 2H); 2.10 (s br, 2H); 1.90-1.75 (m, 2H); 0.95 (t, 3H).

MS (EI; TSQ 700; source 180 C;70 V; 200 uA): 541; 453; 382; 292;291; 247; 219; 106.

Description 13

### (S)-N-(α-ethylbenzyl)-3-[2-(R)-α-aminophenylacetylaminoethoxy]-2-phenylquinoline-4-carboxamide

The reaction was conducted exactly as described in Description 12, utilising the (R)-FMOC-phenylglycinyl chloride instead of the (S). The same amounts of all the reagents were used. 0.8 g of the title compound were obtained.

C₃₅H₃₄N₄O₃

M.P.=92-94° C.

M.W.=558.68

[α]_{D}²⁰=−52.8 (c=0.5. MeOH)

Elemental analysis: Calcd. C, 75.25; H, 6.13; N, 10.03; Found C, 74.15; H, 6.19; N, 9.91.

I.R. (KBr): 3440-3110; 3100-3000; 1670-1630 cm⁻¹.

300 MHz ¹H-NMR (DMSO-d₆): δ 9.30(d, 1H); 8.07 (d, 1H); 7.96 (d, 2H); 7.90 (t br, 1H); 7.72 (m, 1H); 7.60-7.50 (m, 5H); 7.44 (d, 2H); 7.38 (dd, 2H); 7.29-7.19 (m, 6H); 5.09 (dt, 1H); 4.20 (s, 1H); 3.60 (m, 2H); 3.16-2.91 (m, 2H); 2.11 (s br, 2H); 1.90-1.75 (m, 2H); 0.96 (t, 3H).

MS (EI; TSQ 700; source 180 C;70 V; 200 uA): 541; 453; 382; 292; 291; 247; 219; 106.

Description 14

### 2-ethoxycarbonylmethyl-1,2,3,4-tetrahydroisoquinoline

6.0 g (45.0 mmol) of 1,2.3.4-tetrahydroisoquinoline were dissolved, under nitrogen atmosphere, in 60 ml of dry THF. 17.34 g of K₂CO₃ and 5.0 ml (45.2 mmol) of ethyl bromoacetate were added and the reaction mixture was stirred at room temperature overnight. The inorganic salts were filtered off and the solvent was evaporated in vacuo to dryness. The residue was dissolved in CH₂Cl₂ and washed with sat. sol. NaCl, 5% citric acid, sat. sol. NaHCO₃ and sat. sol. NaCl; the organic layer was dried over Na₂SO₄ and evaporated in vacuo to dryness to yield 6.6 g of the title compound which was used without further purification.

C₁₃H₁₇NO₂

M.W.=219.28

I.R. (KBr): 3100-3000; 1752 cm⁻¹.

Description 15

### 2-(2-hydroxyethyl)-1,2,3,4-tetrahydroisoquinoline

1.9 g (50.0 mmol) of LiAlH₄ were suspended, under nitrogen atmosphere, in 100 ml of dry THF; the reaction mixture was cooled at 0° C. and 5.0 g (22.8 mmol) of 2-ethoxycarbonylmethyl-1,2,3,4-tetrahydroisoquinoline (compound of Description 14), dissolved in 100 ml of dry THF, were added dropwise. The reaction was stirred at room temperature for 2 hours, ice-cooled and quenched with 2.5 ml of H₂O, 7.5 ml of 15% NaOH, 2.5 ml of H₂O, stirred for 30 minutes and filtered. The filtrate was evaporated in vacuo to dryness, dissolved in CH₂Cl₂ and washed with sat. sol. NaCl. The organic layer was dried over Na₂SO₄ and evaporated in vacuo to dryness to yield 3.9 g of the title compound which was used without further purification.

C₁₁H₁₅NO

M.W.=177.24

I.R. (KBr): 3700-3100; 3100-3000; 1586 cm⁻¹.

Description 16

### 2-(2-hydroxyethyl)-3,4-dihydro-1(2H)-isoquinolinone

3.8 g (21.4 mmol) of 2-(2-hydroxyethyl)-1,2,3,4-tetrahydroisoquinoline (compound of Description 15), 20.0 g (53.6 mmol) of ethylenediaminetetraacetic acid disodium salt dihydrate and 17.1 g (53.6 mmol) of mercury (II) acetate were dissolved in 95 ml of H₂O. 65 ml of 2N NaOH were added and the reaction was refluxed for 4 hours. After cooling, the reaction was extracted with CH₂Cl₂, washed with 5% HCl, sat. sol. NaHCO₃, sat. sol. NaCl, dried over Na₂SO₄ and evaporated in vacuo to dryness to yield 2.6 g of the title compound which was used without further purification.

C₁₁H₁₃NO,

M.W.=191.23

I.R. (KBr): 3700-3100; 1633; 1604; 1576 cm⁻¹.

300 MHz ¹H-NMR (CDCl₃): δ 8.10 (d, 1H); 7.40-7.10 (m, 3H); 3.90 (s br, 2H); 3.85-3.60 (m, 4H); 3.20 (s br, 1H); 3.05-2.95 (m, 2H).

MS (EI; TSQ 700; source 180 C;70 V; 200 uA): 191 (M+); 173; 160.

Description 17

### 2-(2-chloroethyl)-3,4-dihydro-1(2H)-isoquinolinone

2.5 g (13.1 mmol) of 2-(2-hydroxyethyl)-3,4-dihydro-1(2H)-isoquinolinone (compound of Description 16) were dissolved in 150 ml of CHCl₃. 1.24 ml (17.0 mmol) of SOCl₂, dissolved in 30 ml of CHCl₃, were added dropwise and the reaction mixture was heated to 55° C. for 2 hours and then evaporated in vacuo to dryness. The residue was dissolved in EtOAc, basified with sat sol. K₂CO₃, extracted and washed twice with sat. sol. NaCl. The organic layer was dried over Na₂SO₄ and evaporated in vacuo to dryness to yield 2.7 g of the title compound which was used without further purification.

C₁₁H₁₂ClNO

M.W.=209.67

I.R. (KBr): 3700-3300; 1647; 1605; 1582 cm⁻¹.

300 MHz ¹H-NMR (CDCl₃): δ 810 (d, 1H); 7.45-7.10 (m, 3H); 3.85-3.60 (m. 6H); 3.00 (t, 2H).

MS (EI; TSQ 700; source 180 C;70 V; 200 uA): 209 (M+); 174; 160.

Description 18

### 3-(N-benzyl-N-methylamino)methyl-2-phenylquinoline-4-carboxylic Acid

10.0 g (37.98 mmol) of 3-methyl-2-phenylquinoline-4-carboxylic acid (CAS [43071-45-0]) were dissolved in 500 ml of dichloroethane.

13.7 g (76.12 mmol) of N-bromosuccinimide and 1.0 g (3.85 mmol) of dibenzoyl peroxide were added and the solution refluxed for 8 hours.

The reaction mixture was evaporated in vacuo to dryness and the residue was dissolved in 250 ml of THF; 20 ml (155.50 mmol) of N-benzyl-N-methylamine were added and the solution stirred for 24 hours at room temperature.

The precipitated material was filtered off and the filtrate was evaporated in vacuo to dryness. The residue was dissolved in 300 ml of 10% K₂CO₃ and evaporated in vacuo to dryness. The dark oil was dissolved in 200 ml of acetone, the precipitate was filtered off and the filtrate was evaporated in vacuo to dryness. 100 ml of water were added to the residue and the solution, acidified with 6N HCl, was evaporated in vacuo to dryness.

The residue was dissolved in 28% NH₄OH and the solution was evaporated in vacuo to dryness. The crude product was flash chromatographed on 230-400 mesh silica gel, eluting with a mixture of EtOAc/MeOH 85:15 containing 1.5% of 28% NH₄OH to afford 8.0 g of the title compound as a white solid.

C₂₅H₂₂N₂O₂

M.P.=>250° C.

M.W.=382.46

I.R. (KBr): 3650-3200; 1700; 1660; 1627 cm⁻¹.

300 MHz ¹H-NMR (CDCl₃): δ 8.45 (d, 1H), 8.05 (d, 1H); 7.70-7.05 (m, 12H); 4.20 (s br, 2H); 3.70 (s br, 2H); 3.40 (s br, 1H); 2.00 (s, 3H).

### Example 8

### (R,S)-N-(α-acetylbenzyl)-3-hydroxy-2-phenylquinoline-4-carboxamide

Prepared as described in Description 6 from 0.24 ml (2.8 mmol) of oxalyl chloride, 0.4 ml (5.6 mmol) of DMSO, 0.69 g (1.7 mmol) of (R,S)-N-[α-(1-hydroxyethyl)benzyl]-3-hydroxy-2-phenylquinoline-4-carboxamide (compound of Description 7) and 1.7 ml (12.2 mmol) of TEA.

The work-up of the reaction mixture was carried out in the same manner as described in Description 6. The residue was purified by flash column chromatography on 230-400 mesh silica gel eluting initially with a mixture of petroleum ether/EtOAc 80:20 containing 0.5% NH₄OH (28%) and then with a mixture of petroleum ether/EtOAc 70:30 containing 0.5% NH₄OH (28%) to obtain a crude product which was triturated with i-Pr₂O to yield 96 mg of the title compound as a white solid.

C₂₅H₂₀N₂O

M.P.=163-166° C.

M.W.=396.45

I.R. (KBr): 3400-3000; 1725, 1630, 1570, 1550 cm⁻¹.

300 MHz ¹H-NMR (DMSO-d₆): δ 9.75 (s br, 1H); 9.55 (s br, 1H); 7.95 (m, 3H); 7.82 (m, 1H); 6.60-6.32 (m, 10H); 5.82 (d, 1H); 2.19 (s,

MS (EI; TSQ 700; source 180 C;70 V; 200 uA): 396 (M+); 353; 248; 220; 106.

### Example 9

### (S)-N-(α-ethylbenzyl)-3-(3-phthalimidopropoxy)-2-phenylquinoline-4-carboxamide

4.0 g (10.5 mmol) of (S)-N-(α-ethylbenzyl)-3-hydroxy-2-phenylquinoline-4-carboxamide (product of Description 2) were dissolved in 450 ml of THF.

13.8 g (54.1 mmol) of N-(2-bromopropyl)phthalimide, dissolved in 35 ml of THF, 4.21 g (30.5 mmol) of K₂CO₃ and 0.53 g of KI were added and the suspension was refluxed for 20 hours.

The inorganic salts were filtered off and the reaction mixture evaporated in vacuo to dryness, dissolved in CH₂Cl₂ and washed with water; the organic layer was separated, dried over Na₂SO₄ and evaporated in vacuo to dryness. 2.0 g of the residue were purified by flash column chromatography on 230-400 mesh silica gel, eluting initially with a mixture of hexane/EtOAc 8:2 containing 0.5% NH₄OH (28%) and then with a mixture of hexane/EtOAc 3:2 containing 0.5% NH₄OH (28%). The crude solid so obtained was triturated with i-Pr₂O, filtered, washed and dried to yield 1.1 g of the title compound.

C₃₆H₃₁N₃O₄

M.P.=125-128° C.

M.W.=569.60

[α]_{D}²⁰=−38.2 (c=0.5, MeOH)

Elemental analysis: Calcd. C, 75.91; H. 5.49; N, 7.38; Found C, 75.53; H, 5.50; N, 7.26.

I.R. (KBr): 3400-3120; 3100-3000; 1770; 1740-1700; 1635; 1580 cm⁻¹.

300 MHz ¹H-NMR (DMSO-d₆): δ 9.23 (d, 1H); 8.05 (d, 1H); 7.89 (dd, 2H); 7.86 (m, 4H); 7.72 (ddd, 1H); 7.59 (m, 2H); 7.40 (m, 4H); 7.30 (m, 3H); 7.16 (dd, 1H); 5.03 (dt, 1H); 3.61 (t, 2H); 3.31 (dt, 2H); 1.90-1.58 (m, 4H); 0.96 (t, 3H).

MS (EI; TSQ 700; source 180 C;70 V; 200 uA): 569 (M+); 188; 160.

### Example 10

### (S)-N-(α-ethylbenzyl)-3-{2-[3-(R,S)-hydroxy-3,4dihydro-1(2H)-isoquinolinon-2-yl-ethoxy}-2-phenylquinoline-4-carboxamide (Diastereomeric Mixture)

2.5 g (4.4 mmol) of (S)-N-(α-ethylbenzyl)-3-(2-homophthalimidoethoxy)-2-phenylquinoline-4-carboxamide (compound of Example 6) were dissolved, under nitrogen atmosphere, in 25 ml of MeOH; the solution was cooled to 0° C. and 0.25 g (6.6 mmol) of NaBH₄ were added. The temperature was allowed to raise to room temperature and after 30 minutes additional 0.25 g (6.6 mmol) of NaBH₄ were added and the reaction mixture was maintained under stirring for 1 hour and 15 minutes. Additional 0.5 g (13.2 mmol) of NaBH₄ were added and the reaction mixture was allowed to stand at room temperature overnight. 2 ml of 30% NaOH were added. the organic solvent was evaporated under reduced pressure, and the aqueous solution was extracted with CH₂Cl₂, washed with sat. sol. NaCl, dried over Na₂SO₄ and evaporated in vacuo to dryness. The crude product was purified by gradient flash column chromatography on 230-400 mesh silica gel, utilising a mixture of petroleum ether/EtOAc 7:3 containing 0.5% NH₄OH (28%) as starting eluent and a mixture of petroleum ether/EtOAc 3:7 containing 0.5% NH₄OH (28%) as final eluent.

The crude solid so obtained was triturated with i-Pr₂O, filtered, washed and dried to yield 1.2 g of the title compound.

C₃₆H₃₃N₃O₄

M.P.=100-110° C.

M.W.=571.68

Elemental analysis: Calcd. C, 75.64; H, 5.82; N, 7.35; Found C, 74.44; H, 5.95; N, 7.12.

I.R. (KBr): 3600-3200; 3100-3000; 1732; 1635; 1610; 1580 cm⁻¹.

300 MHz ¹H-NMR (DMSO-d₆): δ 9.29 and 9.25 (d, 1H); 8.05 (d, 1H); 7.92 (m, 2H); 7.86 (dd, 1H); 7.70 (ddd, 1H); 7.56-7.22 (m, 13H); 5.96 and 5.92 (d, 1H); 5.094.84 (m, 2H); 3.99-3.81 (m, 2H); 3.24-3.05 (m, 2H); 2.90-2.80 (m, 2H); 1.90-1.65 (m, 2H); 0.92 and 0.78 (t, 3H).

MS (EI; TSQ 700; source 180 C;70 V; 200 uA): 553; 382; 219; 190; 172.

### Example 11

### (S)-N-(α-ethylbenzyl)-3-(3-aminopropoxy)-2-phenylquinoline-4-carboxamide Hydrochloride

4.1 g (7.4 mmol) of (S)-N-(α-ethylbenzyl)-3-(3-phthalimidopropoxy)-2-phenylquinoline-4-carboxamide (compound of Ex. 9) were dissolved in 200 ml of 96% EtOH and 0.71 ml (13.65 mmol) of hydrazine hydrate were added to the boiling solution. The reaction mixture was refluxed for 24 hours, then additional 0.71 ml (13.65 mmol) of hydrazine hydrate were added and the solution refluxed for 4 hours. After cooling, the reaction mixture was evaporated in vacuo to dryness; 50 ml of H₂O were added and the solution was acidified to pH=1 with 37% HCl. The mixture was refluxed for 1 hour, the insoluble material was filtered off and 30% NaOH was added to pH=10. The solution was extracted with EtOAc, washed with sat. sol. NaCl, dried over Na₂SO₄ and evaporated in vacuo to dryness. The crude product was purified by gradient flash column chromatography on 230-400 mesh silica gel, utilising a mixture of EtOAc/MeOH 95:5 containing 0.5% NH₄OH (28%) as starting eluent and a mixture of EtOAc/MeOH 85:15 containing 0.5% NH₄OH (28%) as final eluent.

The crude solid so obtained was triturated with a warm mixture of i-Pr₂O/EtOAc, filtered, washed and dried to yield 1.4 g of the title compound as a free base. 0.9 g of this free base were dissolved in EtOAc, acidified with HCl/Et₂O, evaporated in vacuo to dryness and triturated with a mixture of EtOAc/acetone to yield 0.8 g of the title compound.

C₂₈H₂₉N₃O₂.HCl

M.P.=160-165° C. dec.

M.W.=476.02

[α]_{D}²⁰=−28.6 (c=0.5, MeOH)

I.R. (KBr): 1653; 1550 cm⁻¹.

300 MHz ¹H-NMR (DMSO-d₆): δ 9.32(d, 1H); 8.08 (d, 1H); 7.92 (in, 2H); 7.80-7.70 (m, 4H); 7.60-7.50 (in, SH); 7.47-7.39 (m, 4H); 7.31 (dd, 1H); 5.08 (dt, 1H); 3.57 (t, 2H); 2.50 (m. 2H); 1.91-1.60 (m, 4H); 0.99 (t, 3H).

MS (EI; TSQ 700; source 180 C;70 V; 200 uA): 439 (M+); 394; 383; 304; 277. 261; 248; 219; 119.

### Example 12

### (S)-N-(α-ethylbenzyl)-3-[2-(1-(2H)-isoquinolinon-2-yl)-ethoxy]-2-phenylquinoline-4-carboxamide

0.8 g (1.4 mmol) of (S)-N-(α-ethylbenzyl)-3-{2-[3-(R,S)-hydroxy-3,4-dihydro-1(2H)-isoquinolinon-2-yl]-ethoxy}-2-phenylquinoline-4-carboxamide (compound of Example 10) were dissolved in 20 ml of dry CH₂Cl₂. The solution was cooled to −10° C., 0.21 ml (1.5 mmol) of TEA were added and a solution of 0.12 ml (1.5 mmol) of methanesulfonyl chloride in 2.5 ml of CH₂Cl₂ was added dropwise. The temperature was allowed to raise to 25° C. and the reaction mixture was stirred overnight. 5 ml of sat. sol. NaHCO₃ were added, the organic layer was extracted, washed with sat. sol. NaCl, dried over Na₂SO₄ and evaporated in vacuo to dryness. The crude product was purified by flash column chromatography on 230-400 mesh silica gel, eluting with a mixture of hexane/EtOAc 7:3 containing 0.5% NH₄OH (28%). The crude solid so obtained was triturated with a warm mixture of i-Pr₂O, filtered, washed and dried to yield 0.4 g of the title compound.

C₃₆H₃₁N₃O₃

M.P.=60° C. dec.

M.W.=553.67

[α]_{D}²⁰=+9.7 (c=0.5, MeOH)

Elemental analysis: Calcd. C, 78.09; H, 5.64; N, 7.59; Found C, 76.86; H, 6.05; N, 7.00.

I.R. (KBr): 3350-3120; 3100-3000; 2968; 2874; 1653; 1594 cm⁻¹.

300 MHz ¹H-NMR (DMSO-d₆): δ 9.29(d, 1H); 8.14 (d, 1H); 8.03 (d, 1H); 7.79-7.68 (m, 5H); 7.60 (m, 2H); 7.52 (dd, 1H); 7.48-7.39 (m, 4H); 7.29 (dd, 1H); 7.11 (dd, 1H); 7.00 (m, 3H); 6.57 (d, 1H); 5.03 (dt, 1H); 3.95-3.74 (m, 4H); 1.89-1.71 (m, 2H); 0.90 (t, 3H).

MS (EI; TSQ 700: source 180 C;70 V; 200 uA): 553 (M+); 249; 172.

### Example 13

### (S)-N-(α-ethylbenzyl)-3-[(S)α-ethylbenzyl]aminomethyl-2-phenylquinoline-4-carboxamide Hydrochloride

5.0 g (15.50 mmol) of t-butyl 3-methyl-2-phenylquinoline-4-carboxylate (obtained by reaction of 3-methyl-2-phenylquinoline-4-carbonyl chloride with t-BuOH), 3.0 g (17.00 mmol) of N-bromosuccinimide and a catalytic amount of dibenzoyl peroxide were dissolved in 100 ml of CCl₄ and the slurry was refluxed for 3 hours.

1.5 g (8.43 mmol) of N-bromosuccinimide were added and the slurry refluxed for additional 2 hours; then, evaporated in vacuo to dryness to yield 11.1 g of a crude material. 1.0 g of this residue was dissolved in 30 ml of abs. EtOH; 1.0 g (7.40 mmol) of (S)-(−)-α-ethylbenzylamine were added and the solution was stirred at room temperature for 1 hour.

The reaction mixture was evaporated in vacuo to dryness. The crude product was purified by gradient chromatography on 70-230 mesh silica gel, eluting with CH₂Cl₂/MeOH (from 0 to 2%) to afford 0.6 g of the title compound as a free base. This was dissolved in Et₂O and the solution acidified with HCl/Et₂O to yield the corresponding hydrochloride. which was recrystallized from EtOAc to obtain 0.25 g of the title compound as a white powder.

C₃₅H₃5N₃O.HCl

M.P.=193-195° C.

M.W.=550.15

[α]_{D}^{°}=−59.8 (c=0.5, MeOH)

Elemental analysis: Calcd. C, 76.41; H, 6.60; N, 7.64; Cl, 6.45; Found C, 76.03; H, 6.66; N. 7.52; Cl, 6.53.

I.R. (KBr): 3441; 3173; 3056; 2968-2582; 1665; 1649; 1539 cm⁻¹.

300 MHz ¹H-NMR (DMSO-d₆, 373K, on the free base): δ 8.88 (d br, 1H); 8.02 (d. 1H); 7.80-7.65 (m, 4H); 7.55-7.28 (m, 9H); 7.20-7.10 (m, 3H); 7.00 (d, 2H); 5.12 (dt, 1H); 4.60 (d, 2H); 3.20 (m, 1H); 2.00-1.80 (m, 3H); 1.65-1.30 (m, 2H); 1.00 (t, 3H); 0.68 (t, 3H).

MS (CI; isobutane gas reagent; P 4000 mTorr; source 150 C): 514(MH+); 394; 379; 349; 136.

### Example 14

### (S)-N-(α-ethylbenzyl)-3-[2-(1,4-dihydro-3(2H)-isoquinolinon-2-yl)ethoxy]-2-phenylquinoline-4-carboxamide

1.2 g (2.1 mmol) of (S)-N-(a:-ethylbenzyl)-3-[2-(2′-hydroxymethylphenylacetyl)aminoethoxy]-2-phenylquinoline-4-carboxamide (compound of Description 8) were dissolved in 30 ml of CHCl₃; HCl/Et₂O was added to pH=4 and a solution of 0.2 ml (2.7 mmol) of SOCl₂ in 6 ml of CHCl₃ was added dropwise. The reaction mixture was warmed to 50° C. for 5 hours; additional 0.1 ml (1.4 mmol) of SOCl₂ were added and the reaction refluxed for 1 hour. The mixture was evaporated in vacuo to dryness, dissolved in EtOAc, washed with sat. sol. K₂CO₃, with sat. sol. NaCl, dried over Na₂SO₄ and evaporated in vacuo to dryness to yield 1.3 g of (S)-N-(α-ethylbenzyl)-3-[2-(2′-chloromethylphenylacetyl)aminoethoxy]-2-phenylquinoline-4-carboxamide as a white solid. This product was dissolved in 25 ml of dry THF and added dropwise to a suspension of 100 mg (4.2 mmol) of NaH in 10 ml of dry THF and 1 ml of 1,3-dimethyl-2-imidazolidinone. The reaction mixture was stirred at room temperature for 4 hours and then quenched with H₂O, evaporated in vacuo to dryness dissolved in EtOAc and washed with sat. sol. NaCl. The organic layer was dried over Na₂SO₄ and evaporated in vacuo to dryness. The crude product was purified by flash column chromatography on 230-400 mesh silica gel, eluting with a mixture of hexane/EtOAc 1:1 to yield 113 mg of the title compound.

C₃₆H₃₃N₃O₃

M.P.=153-156° C.

M.W.=555.68

[α]_{D}²⁰=−20.8 (c=0.5, MeOH)

I.R. (KBr): 3300-3100; 3100-3000; 1660; 1640; 1550 cm⁻¹.

300 MHz ¹H-NMR (DMSO-d₆): δ 9.30 (d, 1H); 8.05 (d, 1H); 7.82 (d, 2H); 7.72 (ddd, 1H); 7.60 (m ,2H); 7.46-7.36 (m, 5H); 7.31-7.22 (m, 6H); 7.16 (m, 1H); 5.05 (dt, 1H); 4.26 (Abq, 2H); 7.80-7.70 (m, 2H); 3.44 (s, 2H); 3.34 (m. 2H); 1.89-1.72 (m, 2H); 0.94 (t, 3H).

MS (EI; TSQ 700; source 180 C:70 V; 200 uA): 382; 264; 247; 219; 172; 119; 91.

### Example 15

### (S)-N-(α-ethylbenzyl)-3-(2-succinimidoethoxy)-2-phenylquinoline-4-carboxamide

0.8 g of (S)-N-(α-ethylbenzyl)-3-[2-(3 carboxypropanoyl)aminoethoxy]-2-phenylquinoline-4-carboxamide (compound of Description 9) and 4 ml of tetrahydronaphthalene were heated to 140° C. for 2.5 hours and, subsequently, to 200° C. for 2 hours. After cooling, 80 ml of EtOAc were added and the solution was washed with sat. sol. NaCl, sat. sol. NAHCO₃, 20% citric acid, sat. sol. NaCl, dried over Na₂SO₄ and evaporated in vacuo to dryness. The residue was purified by flash column chromatography on 230-400 mesh silica gel, eluting with a mixture of hexane/EtOAc 1:1 to yield 148 mg of the title compound.

C₃₁ H₂₉N₃O₄

M.P.=80° C. dec.

M.W.=507.59

[α]_{D}²⁰=−25.4 (c=0.5, MeOH)

I.R. (KBr): 3280; 3100-3000; 1710-1690; 1670-1635; 1530 cm⁻¹.

300 MHz ¹H-NMR (DMSO-d₆): δ 9.29 (d, 1H); 8.05 (d, 1H); 7.84 (dd, 2H); 7.73 (ddd, 1H); 7.58 (m, 2H); 7.56-7.50 (m? 3H); 7.47 (d, 2H); 7.40 (dd, 2H); 7.28 (dd, 1H); 5.08 (dt, 1H); 3.77-3.70 (m, 2H); 3.46-3.32 (m, 2H); 2.54 (s, 4H); 1.90-1.78 (m, 2H); 1.00 (t, 3H).

MS (EI; TSQ 700; source 180 C;70 V; 200 uA): 507 (M+); 478; 374; 221; 126.

### Example 16

### (S)-N-(α-ethylbenzyl)-3-(2-maleimidoethoxy)-2-phenylquinoline-4-carboxamide

0.3 g (5.73 mmol) of (S,Z)-N-(α-ethylbenzyl)-3-[2-(3-carboxypropenoyl)aminoethoxy]-2-phenylquinoline-4-carboxamide (compound of Description 10) were dissolved in 3 ml of acetone. 1.6 ml (11.5 mmol) of TEA were added and the reaction mixture was heated to reflux. 0.82 ml (8.6 mmol) of acetic anhydride were added dropwise to the boiling solution which was refluxed for 22 hours. After cooling, the reaction mixture was poured into ice, stirred for 30 minutes and then extracted with EtOAc. The organic layer was washed with sat. sol. NaCl, 20% citric acid, sat. sol. NaHCO₃ and sat. sol. NaCl, dried over Na₂SO₄ and evaporated in vacuo to dryness. The residue was purified by gradient flash column chromatography on 230-400 mesh silica gel, utilising a mixture of hexane/EtOAc 80:20 as starting eluent and EtOAc as final eluent to yield, after trituration with i-Pr₂O, 100 mg of the title compound.

C₃₁H₂₇N₃O₄

M.P.=74-78° C.

M.W.=505.57

[α]_{D}²⁰=−21.7 (c=0.5, MeOH)

Elemental analysis: Calcd. C, 73.65; H, 5.38; N, 8.31; Found C, 72.50; H, 5.59; N, 7.81.

I.R. (KBr): 3400-3100; 3100-3000; 1710; 1660-1625 cm⁻¹.

300 MHz ¹H-NMR (DMSO-d₆): δ 9.27 (d, 1H); 8.05 (d. 1H); 7.31 (dd, 2H); 7.73 (ddd, 1H); 7.58 (m, 2H); 7.48-7.38 (m, 7H); 7.29 (dd, 1H); 6.95 (s, 2H); 5.05 (dt, 1H); 3.80-3.70 (m, 2H); 3.51-3.35 (m, 2H); 1.88-1.78 (m, 2H); 0.99 (t, 3H).

MS (EI; TSQ 700; source 180 C;70 V; 200 uA): 505 (M+); 476; 372; 220; 124.

### Example 17

### (S)-N-(α-ethylbenzyl)-3-[2-(2,2-dimethyl-4-oxo-3-imidazolidinyl)ethoxy]-2-phenylquinoline-4-carboxamide

0.5 g (1.0 mmol) of (S)-N-(α-ethylbenzyl)-3-(2-aminoacetylaminoethoxy)-2-phenylquinoline-4-carboxamide (compound of Description 11), were dissolved in 100 ml of n-BuOH; 3.5 ml of acetone were added and the reaction mixture was refluxed for 30 hours. The solvent was evaporated in vacuo to dryness and the crude product was purified by gradient flash column chromatography on 230-400 mesh silica gel, utilising a mixture of EtOAc/MeOH 9:1 as starting eluent and a mixture of EtOAc/MeOH 6:4 as final eluent, to yield. after trituration with i-Pr₂O, 0.36 g of the title compound.

C₃₂H₃₄N₄O₃

M.P.=160-162° C.

M.W.=522.65

[α]_{D}²⁰=−50.0 (c=0.5, MeOH)

Elemental analysis: Calcd. C, 73.54; H, 6.56; N, 10.72; Found C, 72.87; H, 6.60; N, 10.63.

I.R. (KBr): 3285; 3100-3000; 1679; 1650-1625; 1587 cm⁻¹.

300 MHz ¹H-NMR (DMSO-d₆): δ 9.28 (d, 1H); 8.06 (d, 1H); 7.93 (dd, 2H); 7.74 (ddd, 1H); 7.61-7.49 (m, 5H); 7.47 (d, 2H); 7.39 (dd, 2H); 7.29 (dd, 1H); 5.10 (dt, 1H); 3.64 (t, 2H); 3.10 (s br, 2H); 3.10-2.90 (m, 2H); 2.79 (s br, 1H); 1.90-1.75 (m, 2H); 1.00 (t, 3H); 1.00 (s, 3H); 0.95(s, 3H).

MS (EI; TSQ 700; source 180 C;70 V; 200 uA): 522 (M+); 383; 360; 248; 141.

### Example 18

### (S)-N-(α-ethylbenzyl)-3-{3-[4-(2-methoxyphenyl)piperazin-1-yl]propoxy}-2-phenylquinoline-4-carboxamide Dihydrochloride

1.0 g (2.6 mmol) of (S)-N-(α-ethylbenzyl)-3-hydroxy-2-phenylquinoline-4-carboxamide (compound of Description 2), 1.0 g (3.7 mmol) of 1-(2-methoxyphenyl)4-(3-chloropropyl)piperazine and 1.6 g (11.7 mmol) of K₂CO₃ were suspended in 20 ml of THF and the reaction mixture was refluxed for 17 hours. Additional 1.1 g (4.1 mmol) of 1-(2-methoxyphenyl)-4-(3-chloropropyl)piperazine and a catalytic amount of KI were added and the reaction refluxed for 4 hours. The inorganic salts were filtered off, the filtrate was evaporated in vacuo to dryness and purified by flash column chromatography on 230-400 mesh silica gel, eluting with a mixture of CH₂Cl₂/MeOH 98:2 containing 0.5% NH₄OH (28%) to obtain 0.6 g of free base which was dissolved in MeOH and acidified to pH=1 with HCl/Et₂O. The solvent was removed in vacuo and the product was triturated with warm EtOAc to yield 0.6 g of the title compound.

C₃₉H₄₂N₄O₃.2HCl

M.P.=151-155° C.

M.W.=687.71

[α]_{D}²⁰=−7.7 (c=0.5, MeOH)

I.R. (KBr): 3600-3300; 3300-3100; 3100-3000; 2800-2000; 1659 cm⁻¹.

300 MHz ¹H-NMR (DMSO-d₆): δ 10.85(s br, 1H); 9.36 (d, 1H); 8.09 (d, 1H); 7.95 (d, 2H); 7.76 (ddd, 1H); 7.66-7.53 (m, 5H); 7.48-7.41 (m, 4H); 7.31 (dd, 1H); 7.08-6.90 (m, 4H); 5.11 (dt, 1H); 3.82 (s, 3H); 3.69 (m, 2H); 3.45 (d br, 2H); 3.28 (dd br, 2H); 3.08-2.89 (m, 4H); 2.86-2.70 (m, 2H); 1.91-1.76 (m, 4H); 1.02 (t, 3H).

MS (EI; TSQ 700; source 180 C;70 V; 200 uA): 614 (M+); 599; 452; 382; 317; 268; 247; 205; 190; 136.

### Example 19

### (S)-N-(α-ethylbenzyl)-3-{2-[2-(R,S)-phenyl-4-oxo-3-imidazolidinyl]ethoxy}-2-phenylquinoline-4-carboxamide (Diastereomeric Mixture)

0.8 g (1.7 mmol) of (S)-N-(α-ethylbenzyl)-3-(2-aminoacetylaminoethoxy)-2-phenylquinoline-4-carboxamide (compound of Description 11) were dissolved in 8 ml of MeOH; 0.25 ml (2.5 mmol) of benzaldehyde were added and the reaction mixture was refluxed for 10 hours. The solvent was evaporated in vacuo to dryness and the residue was purified by gradient flash column chromatography on 230-400 mesh silica gel, utilising a mixture of hexane/EtOAc 1:1 as starting eluent and a mixture of EtOAc/MeOH 9:1 as final eluent, to yield 0.52 g of the title compound.

C₃₆H₃₄N₄O₃

M.P.=80-85° C. dec.

M.W.=570.69

[α]_{D}²⁰=−45.6 (c=0.5, MeOH)

I.R. (KBr): 3400-3120; 3100-3000; 1710-1685; 1680-1650; 1650-1630 cm⁻¹.

300 MHz ¹H-NMR (DMSO-d₆+TFA): δ 9.20 and 9.10 (d. 1H); 8.05 (d, 1H); 7.80-7.70 (m, 3H); 7.60-7.20 (m, 15H); 5.88 and 5.80 (s, 1H); 4.95 (dt, 1H); 4.00 (dd, 1H); 3.85 (dd, 1H); 3.75-3.63 (m, 1H); 3.61-3.40 (m, 3H); 1.80-1.68 (m, 2H); 0.91 and 0.81 (t, 3H).

MS (EI; TSQ 700; source 180 C;70 V; 200 uA): 570 (M+); 467; 435; 408; 383; 334; 305; 264; 247; 219; 189; 118; 91.

### Example 20

### (S)-N-(α-ethylbenzyl)-3-{2-[2,2-dimethyl-5-(S)-phenyl-4-oxo-3-imidazolidiny]ethoxy}-2-phenylquinoline-4-carboxamide

0.5 g (0.9 mmol) of (S)-N-(α-ethylbenzyl)-3-[2-(S)-α-aminophenylacetylaminoethoxy]-2-phenylquinoline-4-carboxamide (compound of Description 12) were dissolved in 10 ml of n-BuOH; 3.5 ml of acetone were added and the reaction mixture was refluxed for 17 hours. The solvent was evaporated in vacuo to dryness and the residue was triturated with i-Pr₂O to yield 440 mg of the title compound.

C₃₈H₃₈N₄O₃

M.P.=167-168° C.

M.W.=598.74

[α]_{D}²⁰=−42² (c=0.5, MeOH)

I.R. (KBr): 3280; 3100-3000; 1690-1670; 1660-1640; 1581 cm⁻¹.

300 MHz ¹H-NMR (DMSO-d₆): δ 9.29 (d, 1H); 8.06 (d, 1H); 7.94 (dd, 2H); 7.73 (ddd, 1H); 7.62-7.20 (m, 15H); 5.09 (dt, 1H); 4.49 (d, 1H); 3.70 (t, 2H); 3.29 (d, 1H); 3.06 (t, 2H); 1.90-1.74 (m, 2H); 1.12 (s, 3H); 1.02 (s, 3H): 0.96 (t, 3H).

MS (EI; TSQ 700; source 180 C;70 V; 200 uA): 598 (M+); 583; 463; 452; 436; 146.

### Example 21

### (S)-N-(α-ethylbenzyl)-3-{2-[2,2-dimethyl-5-(R)-phenyl-4-oxo-3-imidazolidinyl]ethoxy}-2-phenylquinoiine-4-carboxamide

0.5 g (0.9 mmol) of (S)-N-(α-ethylbenzyl)-3-[2-(R)-α-aminophenylacetylaminoethoxy]-2-phenylquinoline-4-carboxamide (compound of Description 13) were dissolved in 10 ml of n-BuOH; 3.5 ml of acetone were added ant the reaction mixture was refluxed for 17 hours. The solvent was evaporated in vacuo to dryness and the residue was purified by gradient flash column chromatography on 230-400 mesh silica gel, utilising a mixture of hexane/EtOAc 1:1 as starting eluent and EtOAc as final eluent, to yield 0.41 g of the title compound.

C₃₈H₃₈N₄O₃

M.P.=147-150° C.

M.W.=598.74

[α]_{D}²⁰=−42.4 (c=0.5, MeOH)

I.R. (KBr): 3272; 3100-3000; 1700-1670; 1660-1630; 1586 cm⁻¹.

300 MHz ¹H-NMR (DMSO-d₆): δ 9.30 (d, 1H); 8.08 (d, 1H); 7.95 (dd. 2H); 7.74 (ddd, 1H); 7.62-7.22 (m, 15H); 5.09 (dt, 1H); 4.46 (d, 1H); 3.78-3.65 (m, 2H); 3.23 (d, 1H); 3.19-3.08 (m, 1H); 3.05-2,93 (m, 1H); 1.90-1.75 (m, 2H); 1.10 (s, 3H); 1.03 (s, 3H); 0.99 (t, 3H).

MS (EI; TSQ 700; source 180 C;70 V; 200 uA): 598 (M+); 583; 463; 452; 436; 146.

### Example 22

### (S)-N-(α-ethylbenzyl)-3-[2-(3,4-dihydro-1(2H)-isoquinolinon-2-yl)ethoxy]-2-phenylquinoline-4-carboxamide

1.0 g (2.61 mmol) of (S)-N-(α-ethylbenzyl)-3-hydroxy-2-phenylquinoline-4-carboxamide (compound of Description 2) were dissolved, under nitrogen atmosphere, in 12 ml of dry THF. 1.1 g of K₂CO₃ and 130 mg of KI were added and then 1.1 g (5.2 mmol) of 2-(2-chloroethyl)-3,4-dihydro-1(2H)-isoquinolinone (compound of Description 17), dissolved in 9 ml of THF, were added dropwise. The reaction was refluxed for 4 hours, filtered and evaporated in vacuo to dryness. The residue was dissolved in CH₂Cl₂ and washed with sat. sol. NaCl; the organic layer was dried over Na₂SO₄ and evaporated in vacuo to dryness. The crude product was purified by gradient flash column chromatography on 230-400 mesh silica gel, utilising a mixture of hexane/EtOAc 1:1 as starting eluent and EtOAc as final eluent, to yield 1.2 g of the title compound.

C₃₆H₃₃N₃O₃

M.P.=71° C. dec.

M.W.=555.67

[α]_{D}²⁰=−24.2 (c=0.5, MeOH)

I.R. (KBr): 3360-3120; 3100-3000; 1660; 1650-1610; 1600; 1580 cm⁻¹.

300 MHz ¹H-NMR (DMSO-d₆): δ 9.29 (d, 1H); 8.05 (d, 1H); 7.90 (d, 2H); 7.84 (d, 1H); 7.71 (ddd, 1H); 7.57 (d, 2H); 7.49 (dd, 1H); 7.44-7.24 (m, 10H); 4.99 (dt, 1H); 3.90-3.78 (m, 2H); 3.60-3.49 (m, 1H); 3.40-3.25 (m, 3H); 2.81 (t, 2H); 1.88-1.67 (m, 2H); 0.87 (t, 3H).

MS (EI; TSQ 700; source 180 C;70 V; 200 uA): 555 (M+); 393; 174.

### Example 23

### (S)-N-(α-ethylbenzyl)-3-(N′-benzyl-N′-methylamino)methyl-2-phenylquinoline-4-carboxamide

8.0 g (20.90 mmol) of 3-(N-benzyl-N-methylamino)methyl-2-phenylquinoline-4-carboxylic acid (compound of Description 18), 5.7 g (41.8 mmol) of (S)-(−)-α-ethylbenzylamine and 5.7 g (41.80 mmol) of HOBT were dissolved in 60 ml of CH₂Cl₂. 11.9 g (57.90 mmol) of DCC dissolved in 20 ml of CH₂Cl₂ were added and the solution was stirred at room temperature overnight.

50 ml of 20% citric acid were added and the solution stirred at room temperature for 2 hours. The precipitated dicyclohexylurea was filtered off and the slurry, basified with solid K₂CO₃, was diluted with 50 ml of H₂O and 50 ml of CH₂Cl₂. The organic phase was separated and the aqueous phase extracted with CH₂Cl₂; the organic phase was dried over Na₂SO₄ and evaporated in vacuo to dryness.

The crude product was flash chromatographed on 230-400 mesh silica gel, eluting with a mixture of hexane/EtOAc 8:2 to afford 4.5 g of crude material which was treated with Et₂O: the precipitated title compound was filtered, triturated with pentane and filtered again to yield 1.6 g of the pure title compound as a white powder.

C₃₄H₃₃N₃O

M.P.=76-78° C.

M.W.=499.65

[α]_{D}²⁰=43.1 (c=1.2 MeOH)

I.R. (KBr): 3290; 3061; 3029; 2970-2789; 1633; 1537 cm⁻¹.

300 MHz ¹H-NMR (DMSO-d₆): δ 8.90 (d, 1H); 8.05 (d, 1H); 7.80-7.05 (m, 16H); 6.85 (d, 2H); 5.15 (m, 1H); 3.75 (s, 2H); 3.15 (s, 2H); 1.90 (m, 2H); 1.65 (s, 3H); 0.95 (t 3H).

MS (EI; TSQ 700; source 180 C;70 V; 200 uA): 408; 380; 273.

### Example 24

### (−)-N-(α-acetylbenzyl)-3-methyl-2-phenyiquinoline-4-carboxamide

3.8 g (10.0 mmol) of (−)-α-aminoacetophenone D-10-camphosulfonate (Benjamin, B. M., Collins, C. J. 1961, J. Am. Chem. Soc., 83, 3662) were dissolved in 1000 mmol of 96% EtOH. 270 mg of PtO₂ were added and the reaction mixture was hydrogenated in a Parr apparatus at 10 psi for 10 minutes. The catalyst was filtered off and the solvent was evaporated in vacuo to dryness to yield 4.0 g of the corresponding 1-amino-1-phenyl-2-propanol D-10-camphosulfonate. 1.5 g (3.9 mmol) of this compound were dissolved in a 1:1 mixture of CH₂Cl₂/CH₃CN; 1.36 ml (9.7 mmol) of TEA were added and the reaction mixture was cooled to −15° C. 1.32 g (4.7 mmol) of 3-methyl-2-phenylquinoline-4-carbonyl chloride (obtained from the corresponding carboxylic acid (CAS [43071-45-0]) by reaction with oxalyl chloride in CH₂Cl₂ at room temperature), dissolved in 50 ml of a 1:4 mixture of CH₂Cl/DMF, were added dropwise, maintaining the temperature below −10° C. The reaction mixture was stirred for 2 hours at 0° C. and then maintained at room temperature overnight. The inorganic salts were filtered off, the filtrate was evaporated in vacuo to dryness, dissolved in CH₂Cl₂ and washed with sat. sol. NaHCO₃, 20% citric acid, sat. sol. NaHCO₃, sat. sol. NaCl. The organic layer was dried over NaSO₄ and evaporated in vacuo to dryness. The crude product was purified by gradient flash column chromatography on 230-400 mesh silica gel, utilising a mixture of CH₂Cl₂/MeOH 99:1 containing 0.5% NH₄OH (28%) as starting eluent and a mixture of CH₂Cl/MeOH 98:2 containing 0.5% NH₄OH (28%) as final eluent, to yield 0.86 g of N-[α-(1-hydroxyethyl)benzyl]-3-methyl-2-phenylquinoline-4-carboxamide. 0.24 ml (2.8 mmol) of oxalyl chloride were dissolved, under nitrogen atmosphere, in 6 ml of dry CH₂Cl₂. The solution was cooled to −55° C. and 0.40 ml (5.6 mmol) of DMSO, dissolved in 1.1 ml of dry CH₂Cl₂, were added dropwise maintaining the temperature below −50° C. The reaction was stirred at −55° C. for 9 minutes, then 0.69 g (1.7 mmol) of N-[α-(1-hydroxyethyl)benzyl]-3-methyl-2-phenylquinoline-4-carboxamide, dissolved in 20 ml of dry CH₂Cl₂, were added keeping the temperature between −50 and −55° C.

After 30 minutes at −55° C., 1.7 ml (12.2 mmol) of TEA were added without exceeding −40° C., then the reaction mixture was allowed to reach room temperature and stirred for additional 15 minutes.

The reaction was quenched with 5 ml of H₂O and extracted with CH₂Cl₂; the organic layer was washed with H₂O, 20% citric acid, sat. sol. NaHCO₃ and brine; the organic layer was separated. dried over Na₂SO₄ and evaporated in vacuo to dryness.

The residual oil was purified by gradient flash column chromatography on 230-400 mesh silica gel, utilising a mixture of petroleum ether/EtOAc 8:2 containing 0.3% NH₄OH (28%) as starting eluent and a mixture of petroleum ether/EtOAc 6:4 containing 0.5% NH₄OH (28%) as final eluent, to yield 0.44 g of the title compound as an amorphous solid.

C₂₆H₂₂N₂O₂

M.P.=55-88° C.

M.W.=394.48

[α]_{D}²⁰=−96.0 (c=0.5 MeOH)

e.e.=74% (chiral HPLC)

I.R. (KBr): 3420-3120; 3100-3000; 1730; 1670-1630; 1580 cm⁻¹.

300 MHz ¹H-NMR (DMSO-d₆): δ 9.51 (d, 1H); 8.00 (d, 1H); 7.81 (m br, 1H); 7.71 (ddd, 1H); 7.58-7.32 (m, 11H); 5.95 (d, 1H); 2.28 (s br, 3H); 2.18 (s, 3H).

MS (EI; TSQ 700; source 180 C;70 V; 200 uA): 394 (M+); 351; 246; 217.

### Example 25

### (+)-N-(α-acetylbenzyl)-3-methyl-2-phenylquinoline-4-carboxamide

Prepared as described in Example 24. 1.69 g of (+)-α-aminoacetophenone hydrochloride (Benjamin. B. M., Collins, C. J., 1961, J. Am. Chem. Soc., 83, 3662) were converted into 1.7 g of the corresponding 1-amino-1-phenyl-2-propanol hydrochloride. 1.6 g (8.5 mmol) of this compound were acylated with 2.9 g (10.2 mmol) of 3-methyl-2-phenylquinoline-4-carbonyl chloride in the presence of 3 ml (21.2 mmol) of TEA to afford 1.9 g of N-[α-(1-hydroxyethyl)benzyl]-3-methyl-2-phenylquinoline-4-carboxamide. 1.9 g (4.8 mmol) of this compound were oxidised in the Swern conditions described in Example 24 (0.7 ml of oxalyl chloride, 1.16 ml of DMSO, 3.5 ml of TEA) to yield 1.4 g of the title compound as an amorphous solid.

C₂₆H₂₂N₂O₂

M.P.=72-95° C.

M.W.=394.48

[α]_{D}²⁰=+83.7 (c=0.5 MeOH)

e.e.=60% (chiral HPLC)

I.R. (KBr): 3420-3120; 3100-3000; 1730; 1670-1630; 1580 cm⁻¹.

300 MHz ¹H-NMR (DMSO-d₆): δ 9.51 (d, 1H); 8.00 (d, 1H); 7.81 (m br, 1H); 7.71 (ddd, 1H); 7.58-7.32 (m, 11H); 5.95 (d, 1H); 2.28 (s br, 3H); 2.18 (s, 3H).

MS (EI; TSQ 700; source 180 C;70 V; 200 uA): 394 (M+); 351; 246; 217.

### Example 26

(R,S)-N-[α-(methoxycarbonyl)-α-(methyl)benzyl]-2phenylquinoline-4-carboxamide

Prepared as described in Description 2 from 1.0 g (4.0 mmol) of 2-phenylquinoline-4-carboxylic acid, 0.9 g (4.2 mmol) of methyl α-methylphenylglicinate hydrochloride [obtained from the corresponding acid (Steinger, R. E., Organic Synthesis. Coll. Vol. 3, 88) by reaction with MeOH and SOCl₂], 1.0 g (7.7 mmol) of HOBT. 0.55 ml (5.0 mmol) of N-methylmorpholine and 0.92 g (4.4 mmol) of DCC in 50 ml of a 2:1 mixture of THF and CH₃CN.

The work-up of the reaction mixture was carried out in the same manner as described in Description 2. The residue was purified by gradient flash column chromatography on 230-400 mesh silica gel, utilising a mixture of petroleum ether/EtOAc 9:1 containing 0.3% NH₄OH (28%) as starting eluent and a mixture of petroleum ether/EtOAc 8:2 containing 0.5% NH₄OH (28%) as final eluent, to yield, after trituration with i-Pr₂O, 38 mg of the title compound.

C₂₆H₂₂N₂O₃

M.P.=154-157° C.

M.W.=410.48

I.R. (KBr): 3400-3100; 3100-3000; 1740; 1660; 1600 cm⁻¹.

300 MHz ¹H-NMR (DMSO-d₆): 6 9.48 (s, 1H); 8.31 (d, 2H); 8.20 (d. 1H); 8.14 (d, 1H); 8.14 (s, 1H); 7.84 (dd, 1H); 7.69 (dd, 1H); 7.63-7.51 (m, 5H); 7.41 (dd, 2H); 7.35 (dd, 1H); 3.77 (s, 3H); 2.0 (s, 3H).

MS (EI; TSQ 700; source 180 C;70 V; 200 uA): 410 (M+); 351; 232; 204.

### Example 27

### (R,S)-N-[α-(methoxycarbonyl)-α-(methyl)benzyl]-3-methyl-2-phenylquinoline-4-carboxamide

5.9 g (27.4 mmol) of methyl α-methylphenylglicinate hydrochloride (see literature reference of Example 26) by reaction with MeOH and SOCl₂) were dissolved in 100 ml of dry Et₂O; 9.6 ml (68.9 mmol) of TEA were added and the solution was cooled to 0° C. 8.6 g (30.4 mmol) of 3-methyl-2-phenylquinoline-4-carbonyl chloride (obtained from the corresponding carboxylic acid (CAS [43071-45-0]) by reaction with oxalyl chloride in CH₂Cl₂ at room temperature), dissolved in 180 ml of a 1:1 mixture of CH₂Cl₂/DMF, were added dropwise maintaining the temperature below 50° C. The reaction was then maintained at room temperature overnight. The solvent was evaporated in vacuo to dryness, the residue was dissolved in CH₂Cl₂ and washed with sat. sol. NaHCO₃, 20% citric acid, sat. sol. NaHCO₃, sat. sol. NaCl. The organic layer was dried over Na₂SO₄, evaporated in vacuo to dryness and purified by gradient flash column chromatography on 230-400 mesh silica gel, utilising a mixture of petroleum ether/EtOAc 8:2 containing 0.3% NH₄OH (28%) as starting eluent and a mixture of petroleum ether/EtOAc 7:3 containing 0.3% NH₄OH (28%) as final eluent, to yield, after trituration with i-Pr₂O, 23 mg of the title compound.

C₂₇H₂₄N₂O₃

M.P.=192-195° C.

M.W.=424.50

I.R. (KBr): 3400-3100; 3100-3000; 1741; 1658 cm⁻¹.

300 MHz ¹H-NMR (DMSO-d₆): δ 9.50 (s, 1H); 8.03 (d, 1H); 7.76 (dd, 1H); 7.68 (dd, 1H); 7.60-7.49 (m, 8H); 7.42-7.31 (m, 3H); 3.78 (s br, 3H); 2.40 (s br, 3H); 2.05 (s br, 3H).

MS (EI; TSQ 700; source 180 C;70 V; 200 uA): 424 (M+); 365; 246; 217.

### Example 28

### (R,S)-N-[at-(acetyl)-α-(methyl)benzyl]-3-methyl-2-phenylquinoline-4-carboxamide

265 mg (0.78 mmol) of Bu₄NHSO₄ were suspended in 1.5 ml of CH₂Cl₂; 250 mg (0.63 mmol) of (R,S)-N-(α-acetylbenzyl)-3-methyl-2-phenylquinoline-4-carboxamide (racemate of Example 24), 0.1 ml (1.6 mmol) of Mel and 0.6 ml of 10% NaOH were added and the reaction mixture was allowed to stand at room temperature overnight. The reaction mixture was washed twice with sat. sol. NH₄Cl and then with sat. sol. NaCl, dried over Na₂SO₄ and evaporated in vacuo to dryness. The residue was dissolved in a 1:1 mixture of hexane/EtOAc and the insoluble inorganic salts were filtered off. The filtrate was evaporated in vacuo to dryness and then purified by gradient flash column chromatography on 230-400 mesh silica gel, utilising a mixture of petroleum ether/EtOAc 8:2 containing 0.3% NH₄OH (28%) as starting eluent and a mixture of petroleum ether/EtOAc 7:3 containing 0.4% NH₄OH (28%) as final eluent, and then by preparative HPLC to yield, after trituration with i-Pr₂O, 17 mg of the title compound.

C₂₇H₂₄N₂O₂

M.P.=167-169° C.

M.W.=408.50

I.R. (KBr): 3290; 3100-3000; 1720; 1641; 1580 cm⁻¹.

300 MHz ¹H-NMR (DMSO-d₆): δ 9.43(s br, 1H); 8.04 (d, 1H); 7.88 (s br, 1H); 7.77 (dd, 1H); 7.67 (dd, 1H); 7.62-7.49 (m, 7H); 7.42 (dd, 2H); 7.34 (dd, 1H); 2.40 (s br, 3H); 2.17 (s, 3H); 2.01 (s, 3H).

MS (EI; TSQ 700; source 180 C;70 V; 200 uA): 408 (M+); 365; 246; 217.

TABLE 1

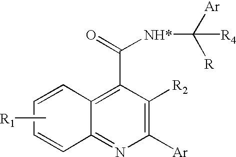

| | | | | | | | Molecular | Melting point | [α]_{D}²⁰ |
|---|---|---|---|---|---|---|---|---|---|
| Ex | Ar | R | R₁ | R₂ | R₄ | * | formula | ° C. | c = 0.5, MeOH |
| 1 | Ph | Et | H | 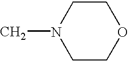 | H | (S) | C₃₀H₃₁N₃O₂.HCl | 173-176 | +11.0 |
| 2 | Ph | Et | H | OCH₂CH₂OH | H | (S) | C₂₇H₂₆N₂O₃ | 129-130 | −41.2 |
| 3 | Ph | Et | 7-Me | OH | H | (S) | C₂₆H₂₄N₂O₂ | 111-114 | — |
| 4 | Ph | Et | H | F | H | (S) | C₂₅H₂₁FH₂O | 67-68 | −22.8 |
| 5 | Ph | Et | H | 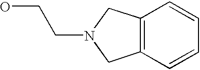 | H | (S) | C₃₅H₃₃N₃O₂.2HCl | 95 dec. | — |
| 6 | Ph | Et | H | 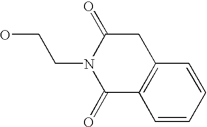 | H | (S) | C₃₆H₃₁N₃O₄ | 159-161 | −29.7 |
| 7 | Ph | Et | H | 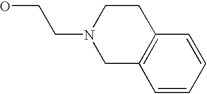 | H | (S) | C₃₆H₃₅N₃O₂.HCl | 120-130 dec. | −14.8 |
| 8 | Ph | Et | H | OH | H | (R, S) | C₂₅H₂₀N₂O₃ | 163-166 | — |
| 9 | Ph | Et | H | 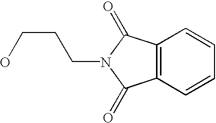 | H | (S) | C₃₆H₃₁N₃O₄ | 125-128 | −38.2 |
| 10 | Ph | Et | H | 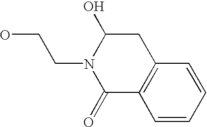 | H | (S) | C₃₆H₃₃N₃O₄ | 100-110 | — |
| 11 | Ph | Et | H | OCH₂CH₂CH₂NH₂ | H | (S) | C₂₈H₂₉N₃O₂.HCl | 160-165 | −28.6 |
| 12 | Ph | Et | H | 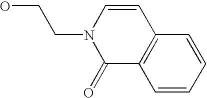 | H | (S) | C₂₆H₃₁N₃O₃ | 60 dec. | +9.7 |
| 13 | Ph | Et | H | (S)CH₂NHCH(Et)Ph | H | (S) | C₃₅H₃₅N₃O.HCl | 193-195 | −59.8 |
| 14 | Ph | Et | H | 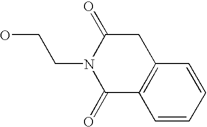 | H | (S) | C₃₆H₃₃N₃O₃ | 153-156 | −20.8 |
| 15 | Ph | Et | H | 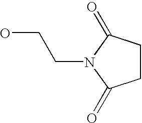 | H | (S) | C₃₁H₂₉N₃O₄ | 80 dec. | −25.4 |
| 16 | Ph | Et | H | 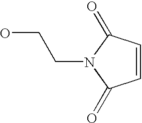 | H | (S) | C₃₁H₂₇N₃O₄ | 74-78 | −21.7 |
| 17 | Ph | Et | H | 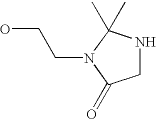 | H | (S) | C₃₂H₃₄N₄O₃ | 160-162 | −50.0 |
| 18 | Ph | Et | H | 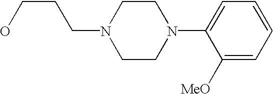 | H | (S) | C₃₉H₄₂N₄O₃.2HCl | 151-155 | −7.7 |
| 19 | Ph | Et | H | 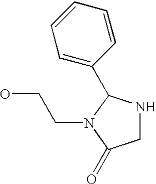 | H | (S) | C₃₆H₃₄N₄O₃ | 80-85 dec. | −45.6 |
| 20 | Ph | Et | H | 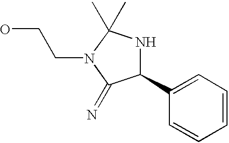 | H | (S) | C₃₈H₃₈N₄O₃ | 167-168 | −42.2 |
| 21 | Ph | Et | H | 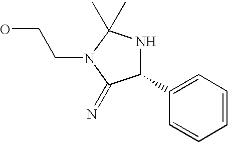 | H | (S) | C₃₈H₃₈N₄O₃ | 147-150 | −42.4 |
| 22 | Ph | Et | H | 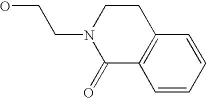 | H | (S) | C₃₆H₃₃N₃O₃ | 71 dec. | −24.2 |
| 23 | Ph | Et | H | CH₂N(CH₃)CH₂Ph | H | (S) | C₃₄H₃₃N₃O | 76-78 | −43.1^{#} |
| 24 | Ph | COCH₃ | H | CH₃ | H | (−) | C₂₆H₂₂N₂O₂ | 55-88 | −96.0 |
| 25 | Ph | COCH₃ | H | CH₃ | H | (+) | C₂₆H₂₂N₂O₂ | 72-95 | +83.7 |
| 26 | Ph | CO₂CH₃ | H | H | CH₃ | (R, S) | C₂₆H₂₂N₂O₃ | 154-157 | — |
| 27 | Ph | CO₂CH₃ | H | CH₃ | CH₃ | (R, S) | C₂₇H₂₄N₂O₃ | 192-195 | — |
| 28 | Ph | COCH₃ | H | CH₃ | CH₃ | (R, S) | C₂₇H₂₄N₂O₂ | 167-169 | — |

^{#}c = 1.2, MeOH

Following synthetic procedures described in Examples 1-28, the compounds listed below have been prepared:

TABLE 2

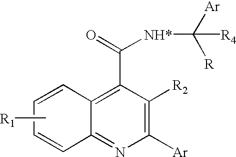

| | | | | | | | | | Melting | [α]_{D}²⁰ |
|---|---|---|---|---|---|---|---|---|---|---|
| | | | | | | | Molecular | Molecular | point | c = |
| Ex | Ar | R | R₁ | R₂ | R₄ | * | formula | weight | ° C. | 0.5, MeOH |
| 29 | Ph | Et | H | CH₂N(CH₃)CH₂CH═CH₂ | H | (S) | C₃₀H₃₁N₃O | 449.590 | 87-88 | −38.4 |
| 30 | Ph | Et | H | 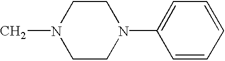 | H | (S) | C₃₆H₃₆N₄O | 540.71 | 142-145 | −44.1 |
| 31 | Ph | Et | H | 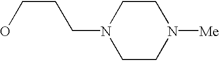 | H | (S) | C₃₃H₃₈N₄O₂.3HCl | 632.070 | 160-170 | −9.6 |
| 32 | Ph | Et | H | 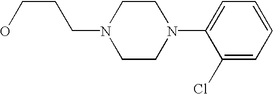 | H | (S) | C₃₈H₃₉ClN₄O₂.2HCl | 692.130 | 120-130 | −1.6 |
| 33 | Ph | Et | H | 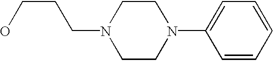 | H | (S) | C₃₈H₄₀N₄O₂.2.5HCl | 675.910 | 86 dec | −7.7 |
| 34 | Ph | Et | H | 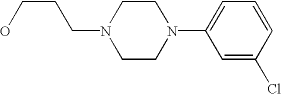 | H | (S) | C₃₈H₃₉ClN₄O₂.2HC | 692.130 | 115-135 | −2.1 |
| 35 | Ph | Et | H | 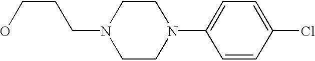 | H | (S) | C₃₈H₃₉ClN₂O₂.2HCl | 692.130 | 128 dec. | −0.6 |
| 36 | Ph | Et | H | 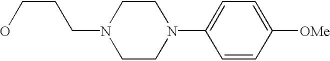 | H | (S) | C₃₉H₄₂N₄O₃.2HCl | 687.714 | 127 dec. | −3.9 |
| 37 | Ph | Et | H | 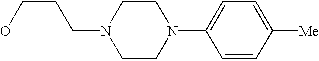 | H | (S) | C₃₉H₄₂N₄O₂.2.5HCl | 689.945 | 160-170 | −6.8 |
| 38 | Ph | Et | H | 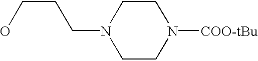 | H | (S) | C₃₇H₄₄N4O₄ | 608.789 | 70-80 | −29.8 |
| 39 | Ph | Et | H | 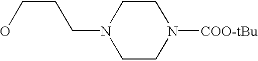 | H | (S) | C₃₂H₃₆N₄O₂.3HCl | 618.044 | 105 dec. | −10.8 |
| 40 | Ph | Et | H | 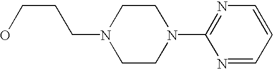 | H | (S) | C₃₆H₃₈N₆O₂.2.5HCl | 677.896 | 140 dec. | −1.9 |
| 41 | Ph | Et | H | 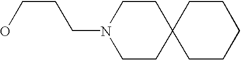 | H | (S) | C₃₈H₄₅N₃O₂.HCl | 612.259 | 140-150 | −8.1 |
| 42 | Ph | Et | H | 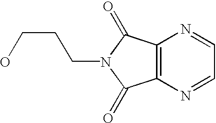 | H | (S) | C₃₄H₂₉N₅O₄ | 571.634 | 100-105 | −32.3 |
| 43 | Ph | Et | H | 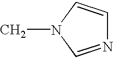 | H | (S) | C₂₉H₂₆N₄O | 446.551 | 232-233 | −23.9 |
| 44 | Ph | Et | H | OCH₂CH₂NHCH₂Ph | H | (S) | C₃₄H₃₃N₃O₂.HCl | 552.110 | 165-169 | −27.7 |
| 45 | Ph | Et | H | OCH₂CH₂N(CH₂Ph)₂ | H | (S) | C₄₁H₃₉N₃O₂.HCl | 642.280 | 144-145 | −25.3 |
| 46 | Ph | Et | H | OCH₂CH₂NHCH₂CH₂Ph | H | (S) | C₃₅H₃₅N₃O₂.HCl | 529.680 | 113-115 | −10.4 |

Table 3. Analytical and spectroscopic data of compounds of Examples 29-46.

| | | | MS (EI; source 200 | |
|---|---|---|---|---|
| Ex. | Elemental analysis | IR (Kbr); cm⁻¹ | ° C.; 70 eV; 200 μA) | 300 MHz¹H NMR (DMSO),303 k |
| 29 | | | 408; 380;273; 261; | (33 K): 8.68(d,1H); 7.72(m,2H; |
| | | | 216; 91. | 7.57-7.42(m,8H; 7.37(dd,2H; |
| | | | | 7.28(dd,1H); 5.40(ddt,1H); |
| | | | | 2.63(d,2H;2.50(s,3H); 2.10-1.82(m,2H); |
| | | | | 0.99(t,3H). |
| 30 | | 3293; 3060-2824; 1633; | 540 (M+); 378: 259; | (353K): 8.84(d br,1H); 8.02(d,1H); |
| | | 1599;1533. | 216; 161; 132; 119; | 7.75(m,2H); 7.60-7.52(m,3H); 7.49-7.42 |
| | | | 105;91; 56. | (m,5H); 7.36(dd,2H); 7.25(dd,1H); 7.19- |
| | | | | 7.12(m,2H); 6.79(d,2H); 6.72(dd,1H); |
| | | | | 5.10(dt,1H); 3.58(s,2H); 2.80(t,4H); 2.21- |
| | | | | 2.10(m,4H); 2.02 1.79(m,2H); 0.98(t,3H). |
| 31 | Calcd. C,62.71; H,6.54; N,8.86; | 3700-3100; 3100-2850; | 522 (M+); 452; 383; | 12.20(s br,2H); 9.37(d,1H); 8.09(d,1H); |
| | Cl,16.83; | 1670-1630;1551. | 139; 113; 91; 70. | 7.92(d,2H); 7.76(ddd,1H): 7.62-7.50 |
| | Found C,56.69; H,6.51; N,7.94; | | | (m,5H); 7.49-7.41(m,4H); 7.32(m,1H); |
| | Cl, 15.06. | | | 5.12(dt,1H); 3.70-3.60(m,4H); 3.60-3.35 |
| | | | | (m,4H); 3.35-3.20(m,2H); 2.81(s,3H); |
| | | | | 2.81-2.60(m,2H); 1.90-1.72(m,4H); |
| | | | | 0.99(t,3H). |
| 32 | Calcd. C,65.94; H,5.97; N,8.09; | 3700-3150; 3150-2800; | 618 (M+); 452; 247; | 11.25(s br,1H); 9.39(d,1H); 8.09(d,1H); |
| | Cl,15.36; | 2750-2000; 1654; 1588; | 209; 119; 91. | 7.95(d,2H); 7.75(ddd,1H); 7.68-7.50 |
| | Found C,65.42; H,6.03; N,7.91; | 1547. | | (m,6H); 7.50-7.40(m,5H); 7.39-7.29 |
| | Cl,13.36. | | | (m,2H); 7.20(d,1H); 7.11(dd,1H); 5.11 |
| | | | | (dt,1H); 3.75-3.63(m,2H); 3.40-3.29 |
| | | | | (m,4H); 3.19(dd,2H); 3.00-2.75(m,4H); |
| | | | | 1.90-1.75(m,4H); 1.01(t,3H). |
| 33 | Calcd. C,67.52; H,6.34; N,8.29; | 3700-3150; 3150-2800; | 584 (M+); 366; 337; | 11.19(s br,1H); 9.39(d,1H); 8.10(d,1H); |
| | Cl,13.11; | 2750-2000; 1658; 1600; | 232; 206; 175. | 7.94(dd,2H); 7.76(ddd,1H); 7.66- |
| | Found C,64.99; H,6.44; N,7.89; | 1538. | | 7.53(m,5H); 7.49-7.40(m,4H); 7.33- |
| | Cl,12.65. | | | 7.26(m,3H); 7.01 (d,2H); 6.88(dd,1H); |
| | | | | 5.10(dt,1H); 3.79-3.63(m,4H); |
| | | | | 3.29(dd,2H); 3.13(dd,2H); 2.95- |
| | | | | 2.82(m,2H); 2.82-2.68(m,2H); 1.91- |
| | | | | 1.75(m,4H); 0.99(t,3H). |
| 34 | Calcd. C,65.94; H,5.97; N,8.09; | 3700-3150; 3150-2800; | 618 (M+); 452; 138; | 11.13(s br,1H); 9.38(d,1H); 8.10(d,1H); |
| | Cl,15.36; | 2750-2000; 1654; 1595; | 104. | 7.98(d,2H); 7.78(ddd,1H); 7.61- |
| | Found C,64.89; H,6.04; N,7.83; | 1539. | | 7.50(m,5H); 7.50-7.40(m,4H); 7.30- |
| | Cl, 13.86. | | | 7.21(m,2H); 7.00(s,1H); 6.95(d,1H); |
| | | | | 6.85(d,2H); 5.10(dt,1H); 3.82(d,2H); 3.72- |
| | | | | 3.62(m,2H); 3,28(dd,2H); 3.19(dd,2H); |
| | | | | 2.90-2.70(m,4H); 1,90-1.70(m,4H); |
| | | | | 0.98(t,3H). |
| 35 | Calcd. C,65.94; H,5.97; N,8.09; | 1650; 1495; 1240. | A) 619 (MH+); 641 | 10.71(s br,1H); 9.37(d,1H); 8.08(d,1H); |
| | Cl,15.36; | | (MNa+); | 7.93(dd,2H); 7.76(ddd,1H); 7.65- |
| | Found C,64.99; H,6.22; N,7.82; | | B (ESI DAU + 619) | 7.52(m,5H); 7.48-7.40(m,4H); 7.33- |
| | Cl, 13.65. | | 237; 210. | 7.28(m,1H); 7.30(d,2H); 7.02(d,2H); |
| | | | | 5.10(dt,1H); 3.78(d,2H); 3.71-3.63 |
| | | | | (m,2H); 3.31(dd,2H); 3.10(dd,2H); 2.95- |
| | | | | 2.70 (m,4H); 1.90-1.75(m,4H); 1.00(t,3H). |
| 36 | Calcd. C,68.11; H,6.45; N,8.15; | 1650; 1450; 1240; | A) 615 (MH+); 637 | 11.00(s br,1H); 9.38(d,1H); 8.09(d,1H); |
| | Cl,10.31; | 1020. | (MNa+); | 7.94(dd,2H); 7.75(ddd,1H); 7.68- |
| | Found C,66.66; H,6.70; N,7.87; | | B (ESI DAU + 615) | 7.52(m,5H); 7.49-7.41 (m,4H); |
| | Cl, 9.78. | | 233. | 7.31(dd, 1H); 6.99(d,2H); 6.89(d,2H); |
| | | | | 5.10(dt,1H); 3.71(s,3H); 3.71-3.65 |
| | | | | (m,2H); 3.60(d,2H); 3.30(dd,2H); |
| | | | | 3.10(dd,2H); 3.00-2.85(m,2H); 2.85- |
| | | | | 2.70(m,2H); 1.90-1.78 (m,4H); 0.99(t,3H). |
| 37 | Calcd. C,67.89; H,6.50; N,8.12; | 1660; 1510; 1440. | A) 599 (MH+); | 10.80(s br,1H); 9.38(d,1H); 8.09(d,1H); |
| | Cl, 12.84; | | B (CID Offset 46 V) | 7.94(dd,2H); 7.76(ddd,1H); 7.65- |
| | Found C,64.53; H,6.65; N,7.53; | | 217; 189. | 7.52(m,5H; 7.48-7.40(m,4H); |
| | Cl,12.95 | | | 7.30(dd,1H); 7.09(d,2H); 6.90(d,2H); |
| | | | | 5.10(dt,1H); 3.75-3.62(m,4H); |
| | | | | 3.29(dt,1H); 3.05(dd,1H); 2.97- |
| | | | | 2.70(m,6H); 2.23(s,3H); 1.90-1.75(m,4H); |
| | | | | 0.99(t,3H). |
| 38 | | 3290; 2970; 1690; | A) 609 (MH+); 631 | 9.28(d,1H); 8.06(d,1H); 7.92(dd,2H); |
| | | 1640; 1530; 1420; | (MNa+) | 7.72(ddd,1H); 7.63-7.50(m,5H); |
| | | 1170. | | 7.45(d,2H); 7.38(dd,2H); 7.28(dd,1H); |
| | | | | 5.09(dt,1H); 3.69-3.58(m,2H); |
| | | | | 3.17(m,4H); 2.01(m,6H); 1.89-1.74 |
| | | | | (m,2H); 1.51-1.41(m,2H); 1.39(s,9H); |
| | | | | 0.90(t,3H). |
| 39 | Calcd. C,62.18; H,6.36; N,9.06; | 1650; 1450; 1300. | A) 509 (MH+); 531 | 11.99(s br,1H); 10.09(s br,1H); 9.89(s |
| | Cl,17.21; | | (MNa+); | br,1H); 9.38(d,1H); 8.09(d,1H); |
| | Found C,57.72; H,6.58; N,8.31; | | B (ESI DAU + 509) | 7.92(dd,2H); 7.75 (ddd,1H); 7.64- |
| | Cl,16.11. | | 127. | 7.55(m,5H); 7.48-7.41(m,4H); |
| | | | | 7.32(m,1H); 5.10(dt,1H); 3.72-3.62 |
| | | | | (m,2H); 3.53-3.30(m,6H); 3.30- |
| | | | | 3.05(m,2H); 2.82-2.62 (m,2H); 1.91- |
| | | | | 1.75(m,4H); 0.99(t,3H). |
| 40 | Calcd. C,63.78; H,6.02; | 1660; 1540; 1350. | A) 587 (MH+); 609 | 11.30(s br,1H); 9.38(d,1H); 8.49(d,2H); |
| | N,12.40; Cl,13.07; | | (MNa+); | 8.09(d,1H); 7.92(dd,2H); 7.75(ddd,1H); |
| | Found C,60.79; H,6.46; | | B (ESI DAU + 587) | 7.65-7.50(m,5H); 7.48-7.38(m,4H); |
| | N,11.81; Cl,13.10. | | 205. | 7.27(dd,1H); 6,79(dd,1H); 5.10(dt,1H); |
| | | | | 5.65(d,2H); 3.75-3.62 (m,2H); |
| | | | | 3.39(dd,2H); 3.29(dd,2H); 2.81-2.65 |
| | | | | (m,4H); 1.90-1.75(m,4H); 0.99(t,3H). |
| 41 | | 1650; 15501450; 1300. | A) 576 (MH+); | 10.19(s br,1H); 9.35(d,1H); 8.09(d,1H); |
| | | | B (ESI DAU + 576) | 7.93(dd,2H); 7.75(ddd,1H); 7.65- |
| | | | 194; 166. | 7.53(m,5H); 7.47-7.39(m,4H); |
| | | | | 7.30(dd,1H); 5.10(dt,1H); 3.72- |
| | | | | 3.60(m,2H); 2.99(dd,2H); 2.79- |
| | | | | 2.62(m,4H); 1.88-1.72(m,4H); 1.68(d,2H); |
| | | | | 1.53(ddd,2H); 1.45-1.35(m,8H); |
| | | | | 1.22(m,2H); 0.99(t,3H). |
| 42 | | *3280; 1728; 1660- | 219; 190; 163 | 9.27(d,1H); 9.01(s,2H); 8.06(d,1H); |
| | | 1640. | | 7.91(d,2H); 7.71(ddd,1H); 7.58(m,2H); |
| | | | | 7.48-7.31(m,7H); 7.21(dd,1H); |
| | | | | 5.08(dt,1H); 3.69(t,2H); 3.51-3.35 (m,2H); |
| | | | | 1.90-1.69(m,4H); 0.97(t,3H). |
| 43 | | 3230; 1660; 1550. | A) 447 (MH+); | 9.50 (2d,1H); 7.70-8.10 (m,3H); 7.10-7.55 |
| | | | B (ESI DAU + 447) | (m,11H); 6.48-6.90 (m,3H); 5.30 (s,1H); |
| | | | 261; 119; 91. | 4.85-5.15 (m,2H); 1.65-1.95 (m,2H); 0.90 |
| | | | | (2t,3H). |
| 44 | | 3498; 3185; 2968-2637; | 408; 273; 380. | 8.89 (d,1H); 8.01 (d,1H); 7.74 (m,2H); |
| | | 1650; 1535. | | 7.62 (dd,2H); 7.57-7.44 (m, 6H); 7.39 |
| | | | | (dd,2H); 7.29 (dd,1H); 7.20-7.10 (m, |
| | | | | 3H); 6.89 (m,2H); 5.13 (dt,1H); 3.70 (s, |
| | | | | 2H); 3.10 (s,2H); 2.02-1.80 (m,2H); 1.68 |
| | | | | (s,3H); 0.98 (t,3H). |
| 45 | | 3419; 3163; 3059-2933; | 514; 223; 210; 132; | 9.52 (d,1H); 8.10 (d,1H); 7.86 (dd,2H); |
| | | 1656; 1542. | 91. | 7.79 (ddd,1H); 7.63 (m,2H); 7.49-7.36 |
| | | | | (m, 16H); 7.30-7.20 (m, 3H); 5.01 (dt, |
| | | | | 1H); 4.09 (m, 4H); 3.99 (m, 2H); 3.00 (m, |
| | | | | 2H); 1.81-1.71 (m,2H); 0.82 (t,3H). |
| 46 | | 3388; 2930; 1630; | 438; 383; 320; 303; | 9.48(d,1H); 8.91(s br,1H); 8.09(d,1H); |
| | | 1563. | 291; 247; 219; 204; | 7.98(dd,2H); 7.76(ddd,1H); 7.61(m,2H); |
| | | | 119; 105; 91; 56. | 7.58-7.50(m,3H); 7.48-7.25(m,8H); |
| | | | | 7.21(d,2H); 5.07(dt,1H); 3.98-3.85 |
| | | | | (m,2H); 2.85(s br,6H); 1.90-1.74(m,2H); |
| | | | | 0.93(t,3H). |

*Nujol moul. A) ESI POS; TSQ 700; solvent: methanol/spray:4.5 kV/skimmer: 60 eV/capillary 220° C.

### Table 4. Pharmacological Data

| | Binding affinity in hNK-3-CHO^{a} |
|---|---|
| Example n. | IC₅₀ (nM) |
| 2 | 1.6 |
| 5 | 1.2 |
| 6 | 0.8 |
| 9 | 3.2 |
| 11 | 2.6 |
| 14 | 1.7 |
| 17 | 3.4 |
| 18 | 0.4 |
| 21 | 0.9 |
| 22 | 1.3 |
| 30 | 1.1 |
| 31 | 3.3 |
| 33 | 0.7 |
| 34 | 0.8 |
| 40 | 1.1 |
| 42 | 2.7 |

^{a}hNK-3-CHO = human neurokinin-3 receptors expressed in CHO cell lines; radioligand used was [¹²⁵I]-[Me-Phe⁷]-NKB.

1. A compound of formula (I): 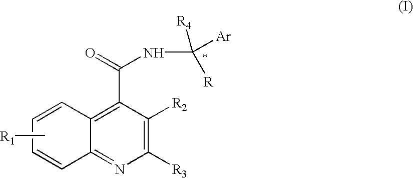
or a salt thereof, or a solvate thereof, wherein, Ar is an optionally substituted aryl or a C₅₋₇ cycloalkdienyl group, or an optionally substituted single or fused ring aromatic heterocyclic group,;
R is C₁₋₆ alkyl, C₁₋₇ cycloalkyl, C₃₋₇ cycloalkylalkyl, optionally substituted phenyl or phenyl C₁₋₆ alkyl an optionally substituted five-membered heteroaromatic ring comprising up to four heteroatoms selected from O and N, hydroxy C₁₋₆ alkyl, amino C₁₋₆ alkyl, C₁₋₆ alkylaminoalkyl, di C₁-6 alkylaminoalkyl, C₁₋₆ acylaminoalkyl, C₁₋₆ alkoxyalkyl, C₁₋₆ alkylcarbonyl, carboxy, C₁₋₆ alkoxycarbonyl, C₁₋₆ alkoxycarbonyl C₁₋₆ alkyl, aminocarbonyl, C₁₋₆ alkylaminocarbonyl, di C₁₋₆ alkylaminocarbonyl, halogeno C₁₋₆ alkyl; or R is a group —(CH₂)ₚ— wherein p is 2 or 3 which group forms a ring with a carbon atom of Ar;
R₁ represents hydrogen or up to four optional subtitutents selected from the list consisting of: C₁₋₆ alkyl, C₁₋₆ alkenyl, aryl, C₁₋₆ alkoxy, hydroxy, halogen, nitro, cyano, carboxy, carboxamido, sulphonamido, C₁₋₆ alkoxycarbonyl, trifluoromethyl, acyloxy, phthalimido, amino or mono- and di-C₁₋₆ alkylamino;
R₂ represents hydrogen, C₁₋₆-alkyl, hydroxy, halogen, cyano, amino, mono- or di-C₁₋₆-alkylamino, alkylsulphonylamino, mono- or di-C₁₋₆-alkanoylamino wherein any alkyl group is optionally substituted with an amino group or with a mono- or di-alkylamino group, or R₂ is a moiety —X—(CH₂)ₙ—Y wherein X is a bond or —O— and n is an integer in the range of from 1 to 5 providing that when X is —O— n is only an integer from 2 to 5 and Y represents a group NY₁ Y₂ wherein Y₁ and Y₂ are independently selected from hydrogen, C₁₋₆-alkyl, C₁₋₆-alkenyl, aryl or aryl-C₁₋₆-alkyl or Y is hydroxy, halogen or an optionally substituted N-linked single or fused ring, heterocyclic group,
R₃ is branched or linear C₁₋₆ alkyl, C₃₋₇ cycloalkyl, C₄₋₇ cycloalkylalkyl, optionally substituted aryl, or an optionally substituted single or fused ring aromatic heterocyclic group; and
R₄ represents hydrogen or C₁₋₆ alkyl:

2. A compound according to claim 1, wherein Ar represents phenyl.

3. A compound according to claim 1 or claim 2, wherein R represents ethyl.

4. A compound according to any one of claims 1 to 3, wherein R₂ represents a moiety —X—(CH₂)ₙ—Y.

5. A compound according to any one of claims 1 to 4, wherein the moiety —X—(CH₂)ₙ—Y is a moiety of formula (a): 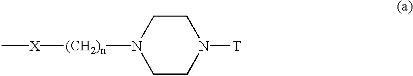
wherein T represents C₁₋₆ alkyl, C₁₋₆ alkoxycarbonyl, aryl or an aromatic heterocyclic group and either X is O and n is 2 or 3 or X is a bond and n is 1, 2 or 3.

6. A compound according to claim 5, wherein T represents a methyl group.

7. A compound according to claim 5, wherein T represents a phenyl group, substituted with one or more alkoxy groups.

8. A compound according to claim 5, wherein T represents a pyrimidine group.

9. A compound according to claim 1, wherein —X—(CH₂)ₙ—Y is a moiety of formula (b): 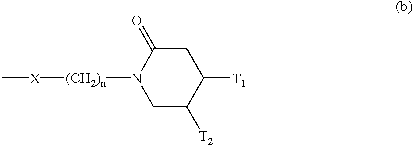
wherein X is O or a bond, n is 1, 2 or 3, TI and To each independently represents hydroxy, C₁₋₆ alkoxycarbonyl, C₁₋₆ alkyl, aryl or a single or fused ring aromatic heterocyclic group, or T₁ and T₂ together with the carbon atoms to which they are attached form a carbocyclic ring; said aryl or aromatic heterocyclic groups being optionally substituted with one or two C₁₋₆ alkyl, alkoxy, hydroxy, halogen, halogenalkyl groups; or one of T₁ or T₂ is an oxo group and the other is selected from the above mentioned groups as appropriate.

10. A compound according to claim 9, wherein T₁ and T₂ together with the carbon atoms to which they are attached form a carbocyclic ring.

11. A compound according to claim 9, wherein R₂ represents n is an integer 1 or 2.

12. A compound according to claim 1, wherein:
Ar is phenyl, R is ethyl, R₁ is hydrogen, R₂ is a moiety —X—(CH₂)ₙ—Y wherein X is O n is 1, 2 or 3 and Y is a moiety formula (a) as defined in claim 5 or a moiety of formula (b) as defined abovein claim 9.

13. A compound according to claim 1 as described in Examples 1-46 herein, or a salt thereof, or a solvate thereof

14. A compound according to claim 1 as described in Examples 18, 30, 33 and 40 herein, or a salt thereof, or a solvate thereof

15. A process for the preparation of a compound of formula (I), or a salt thereof and/or a solvate thereof, which process comprises reacting a compound of formula (III): 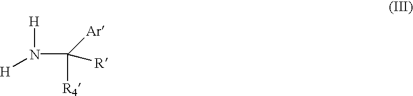
wherein R′, R₄′ and Ar′ are R, R₄ and Ar as defined for formula (I) or a group or atom convertible to R, R₄ and Ar respectively, with a compound of formula (II) or an active derivative thereof: 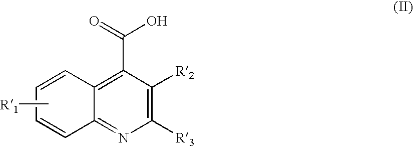
wherein R′₁, R′₂ and R′₃ are R₁, R₂ and R₃ respectively as defined in relation to formula (I) or a group convertible to R₁, R₂ and R₃ to form a compound of formula (Ib): 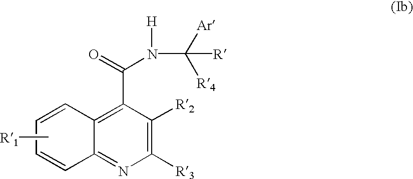
wherein Ar′, R′, R′₁, R′₂, R′₃and R′₄ are as defined above, and optionally thereafter carrying out one or more of the following optional steps:
(i) converting any one of Ar′, R′, R′₁, R′₂, R′₃ and R′₄ to Ar, R, RI, R₂, , R₃ or R₄ respectively as required, to obtain a compound of formula (I);
(ii) converting a compound of formula (I) into another compound of formula (I); and
(iii) preparing a salt of the compound of formula (I) and/or a solvate thereof.

16. A pharmaceutical composition comprising a compound of formula (I), or a pharmaceutically acceptable salt thereof, or a pharmaceutcally acceptable solvate thereof and a pharmaceutically acceptable carrier.

17. A method for the treatment and/or prophylaxis of the Primary and Secondary Conditions in mammals, which method comprises administering to the mammal in need of such treatment and/or prophylaxis an effective, non-toxic amount of a compound of formula (I) or a pharmaceutically acceptable salt thereof, or a pharmaceutically acceptable solvate thereof.

18. A compound of formula (I), or a pharmaceutically acceptable salt thereof or a pharmaceutically acceptable solvate thereof, for use as an active therapeutic substance.

19. A compound of formula (I), or a pharmaceutically acceptable salt thereof or a pharmaceutically acceptable solvate thereof, for use for the treatment and/or prophylaxis of Primary and Secondary Conditions.

20. The use of a compound of formula (I), or a pharmaceutically acceptable salt thereof or a pharmaceutically acceptable solvate thereof, in the manufacture of a medicament for the treatment of the Primary and Secondary Conditions.
